(19) United States
(12) Patent Application Publication
(10) Pub. No.: US 2012/0095322 A1
(43) Pub. Date: Apr. 19, 2012

(54) Devices, systems and methods for multimodal biosensing and imaging

(76) Inventors: Nikolaos V. Tsekos, Houston, TX (US); Ahmet E. Sonmez, Houston, TX (US)

(21) Appl. No.: 13/199,741

(22) Filed: Sep. 8, 2011

Related U.S. Application Data
(60) Provisional application No. 61/402,941, filed on Sep. 8, 2010.

(51) Int. Cl. A61B 6/00; A61B 8/13; A61M 5/32; A61B 10/02; A61B 6/03; A61M 25/00; A61B 6/02; A61B 5/055

(52) U.S. Cl. 600/411; 600/407; 600/425; 600/476; 600/437; 600/410; 600/427; 600/436; 600/431; 600/433

(57) ABSTRACT

Provided herein are robotic or automated multimodal tissue scanning or imaging systems and methods. The systems comprise a robotic delivery device configured to mechanically scan one or more zones of a tissue by co-registering at least two modalities, i.e., one or more of each of a sensor modality and an imaging modality and acquiring sensor modality data at one or more positions in the zone(s) as the sensor modality is dimensionally translated along the device axes. The system also comprises a computational core of a plurality of interlinked modules for planning, processing, tissue-sampling, visualization, fusion, and control of the delivery device and the system. The methods provided herein utilize the automated systems to produce spatial maps based on the analyzed sensor modality data.

### CROSS-REFERENCES TO RELATED APPLICATIONS

This non-provisional application claims benefit of provisional application U.S. Ser. No. 61/402,941, filed Sep. 8, 2010, now abandoned, the entirety of which is hereby incorporated by reference.

### GOVERNMENTAL SPONSORSHIP

The U.S. Government has a paid-up license in this invention and the rights in limited circumstances to require the patent owners to license others on reasonable terms as provided for by the terms of grant No. CNS-0932272 awarded by the National Science Foundation.

### BACKGROUND OF THE INVENTION

1. Field of the Invention

The present invention relates to the field of medical imaging, particularly, multimodality imaging, and robotics. Specifically, the present invention relates to a method, device and system for performing multimodality imaging and/or biosensing for assessing the pathophysiology of tissue in situ for diagnostic, therapeutic, and interventional, including surgical, procedures, by means of an actuated or robotic device that mechanically scans the area of interest by carrying one or multiple Limited-FOV sensors.

2. Description of the Related Art

Recent advances in understanding the molecular features of lesions in vivo and the rapid evolution of the associated molecular imaging modalities offer new opportunities in patient care (1-14, 32-36). A wide range of modalities have been introduced, or traditional ones adopted, to assess regional biochemistry and morphology, including radionuclide imaging, magnetic resonance imaging (MRI) and spectroscopy (MRS), optical imaging and contrast enhanced ultrasound (11-12, 15, 33-34, 37). Endowed with the capability of those modalities to assay biochemical processes at the molecular level and assess morphology at the cellular level, the emerging “molecular medicine” may produce new in situ diagnostic and therapeutic approaches (1-36).

The biomolecular or functional processes and structural features associated with the healthy and diseased tissue are complex and occur, or their consequences manifested, at different levels of the living matter. Traditionally, patient management entails the assessment of pathophysiology at several such levels. An example is the combination of laboratory tests, histology of biopsy samples, and imaging. If, hypothetically, that multi-level information can be collected in situ and interpreted accurately and rapidly, then patient management may improve by reducing time, cost and the consequences to the patients, their families, the health care system and the society. It becomes evident from the literature (1-36) that the advent of the aforementioned molecular/cellular modalities, combined with traditional ones, may enhance our ability to probe at three levels: molecular, cellular or near-cellular, and tissue (macroscopic). Until recently, the majority of such approaches were primarily used to study cultures, extracted tissue or small animal models. A bench-to-bedside translation of such assaying methods is an active field of research and development.

Multimodality approaches have been and are pursued for the collection of complementary information that assay different aspects of pathophysiology (46-52). Among those multimodal approaches, characteristic examples are the combination of contrast enhanced MRI with localized proton MRS (53-59), MRI with whole-breast diffuse optical tomography (DOT) (2, 38-40, 60-69) [2, 38-40, 60-69], and endoscope-based OCT and fluorescence in the assessment of oral (70), ovarian (71) and colon (72-73) cancers.

To probe at the molecular level, and assess non-destructively biochemical processes, in this invention it is disclosed the use of modalities that sense tissue in the molecular level such as MR spectroscopy (MRS), light induced fluorescence (LIF), confocal microscopy (CoM) and the same. It should be emphasized that, while MRS probes molecular species and events, its spatial resolution is not at the molecular level. MRS has the ability to characterize the biochemical contents of tissue in vivo (37, 51, 74-99), by sensing a wide range of biologically relevant nuclei, such as hydrogen (¹H), phosphorous (³¹P) and carbon (¹³C). In many cases, MRS can measure the local concentration of biochemical species allowing the assessment of metabolic and cellular processes. In breast cancer, in vivo 1H-MRS studies have shown that a resonance at a chemical shift of 3.2 ppm is observed in malignant but not in benign or normal tissue (53-59, 91-106) [53-59, 91-105]. This resonance is primarily attributed to Choline-containing compounds and is a superposition of resonances from several species including free Choline, phosphocholine, and Choline head groups on lipids (106-109).

In vivo these resonances are broadened and indistinguishable, even at 4 Tesla, and this peak is referred to as the “total Choline” (tCho) (56, 110). The exact mechanism(s) that elevate tCho are not fully identified; a hypothesis is that elevated tCho is related to increased cellular proliferation and membrane turnover, although tCho may also increase due to changes in biosynthesis and/or catabolism. Nevertheless, studies have shown that tCho is an indicator of malignancy, as well as that tCho decreases or disappears in response to chemotherapy (94). Although MRS can sense those species non-invasively and without contrast agents, it has limited spatial resolution (order of 1 cm) primarily due to the inherent low sensitivity of the nuclear magnetic resonance (NMR).

To probe at the cellular or near-cellular level and assess microscopic morphology in vivo, in this invention we disclose the use of Optical Coherence Tomography (OCT) and the same. Optical methods have demonstrated ability for assessing morphological and biochemical processes (22, 111-117), using the inherent optical properties of the tissue, as well as optical contrast agents (43, 45). Among those optical methods, OCT, which detects near-infrared (NIR) reflections on large and undifferentiated cells, offers near-cellular level resolution at the range of <20 μm (5, 73, 118-123), while modern OCT scanners used in ophthalmology have resolution <2 μm. OCT has demonstrated a high correlation with tissue histopathology (e.g. in breast cancer (5, 73, 118-120) and its capabilities may be further enhanced with the use of exogenous optical contrast agent (11, 14, 43, 45). However, a major drawback of OCT, common to the high resolution or high sensitivity optical methods, is limited tissue penetration to about 2 mm. To address this limitation in situ practice of OCT requires (73, 118-120, 124-131): an invasive fiberoptic or endoscopic approach, i.e. trans-cannula, to reach a targeted lesion, and guidance with a tissue level modality, e.g. x-ray based, ultrasound or MRI) to detect the lesion and guide the OCT probe.

To probe at the tissue at the macroscopic level, this invention discloses the use of magnetic resonance imaging (MRI). While this choice is dictated by the selection of MRS, MRI offers certain benefits: a) Plethora of contrast mechanisms to assess the physiology of the lesion(s) and the surrounding tissue, such as perfusion (16, 132-145) and angiography (48, 50, 147-148), b) High spatial resolution, c) true three-dimensional (3D) or multislice imaging, d) Operator independent performance (vs. ultrasound), and e) lack of ionizing radiation vs. CT or mammography. With its 3D acquisition the modality is ideal for guiding an interventional tool such as the OCT probe. Alternatively, this invention discloses the use of computed tomography (CT), or 2D or 3D ultrasound (US).

Therefore, there is a recognized need in the art for improved in situ diagnostic and therapeutic approaches even with the current advances in understanding the molecular features of lesions in vivo that may lead to assessing the malignancy of a tumor in situ. Thus, the prior art is deficient the methods, device and system for multimodality and multi-level imaging, image-guided scanning and spectroscopy in situ. The present invention fulfills this longstanding need and desire in the art.

### SUMMARY OF THE INVENTION

The present invention is directed to an automated system for multimodality imaging of a tissue in a subject. The system comprises a robotic delivery device configured for mechanically scanning the area of interest, a computational core comprising a plurality of software modules electronically interlinked with the delivery device, one or more interfaces between one or both of the computational core and the delivery device, at least one limited field of view sensor modality mechanically linked to or carried on the robotic delivery device and electronically linked to the computational core, and an operator thereof, and at least one wide field of view imaging modality electronically linked to the computational core. In a related automated systems, the robotic delivery device may further comprise a medium for coupling the one or more sensor modalities to the tissue and/or means for image-guided tissue sampling mechanically linked to or carried on the robotic delivery device and electronically linked to the computational core. In another related automated system the robotic delivery device may further comprise one or more channels configured to accommodate means for delivery associated with a type of the device, to deliver locally one or more therapeutic agents, one or more diagnostic agents or a combination thereof, or to deliver locally one or more contrast agents compatible with the sensor modality, the imaging modality, or to remove one or more fluids obstructing the link between the sensor modality and the tissue.

The present invention also is directed to a method for multimodal image scanning of a tissue. The method comprises the steps of selecting at least two modalities and co-registering the same and selecting an area of interest on or in the tissue. The area of interest is scanned via the co-registered modalities and the multimodal data collected during scanning is analyzed. A related method further comprises obtaining a tissue sample at the area of interest for analysis. Another related method further comprises delivering locally one or more of a therapeutic, diagnostic or contrast agent prior to or during scanning. Another related method further comprises visually displaying the analyzed data as one or more spatial maps. Yet another related method further comprises diagnosing a pathophysiological condition based on the analyzed multimodal data.

The present invention is directed further to a multimodal tissue scanning system. The system comprises a robotic delivery device configured to deliver a scanning signal from at least one sensor modality to one or more scanning zones on or in the tissue and to collect data during scanning where the one or more scanning zones are determined from one or more selected imaging modalities. The robotic delivery device is electronically linked to a planning module configured to plan collection of multimodal data during scanning, a processing module configured to analyze the collected data, a visualization module configured to graphically output the analyzed data, a fusion module configured to co-register all the modalities and co-visualize the analyzed scanning data, and a control module configured to link the delivery device to the modules. A related system further comprises means for image-guided tissue sampling mechanically linked to or carried on the robotic delivery device and electronically linked to a tissue sampling module configured to enable sampling or biopsy of the tissue at the one or more scanning zones. Another related system further comprises a medium for coupling the scanning signal to the tissue, a channel to deliver locally to the tissue one or more therapeutic agents, one or more diagnostic agents or a combination thereof or a channel to locally deliver to the tissue one or more contrast agents compatible with the one or more modalities.

Other and further aspects, features, benefits, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions and certain embodiments of the invention briefly summarized above are illustrated in the appended drawings. These drawings form a part of the specification. It is to be noted, however, that the appended drawings illustrate preferred embodiments of the invention and therefore are not to be considered limiting in their scope.

FIG. 1 is an overview of the system, processes and paths of data and control flow depicting the two primary elements, the computational core and the delivery device, of the RoboScanner system.

FIG. 2 is an overview of the RoboScanner computational core software illustrating the primary Planning, Control, Processing and Visualization modules. Each Module includes a multitude of synergizing routines for the different tasks that each module performs.

FIG. 3 is an overview of the hardware components and interfacing of the RoboScanner system.

FIGS. 4A-4D illustrates the general architecture of the RoboScanner device, depicting its primary elements (FIG. 4A) and probe designs (FIG. 4B-4D).

FIGS. 5A-5B depict a flowchart showing the operation of the RoboScanner system delineating the tasks and the module related to each of the particular tasks.

FIGS. 6A-6G are a diagrammatic description of the planning tasks of the operation of the RoboScanner including setting the P Trajectory (FIG. 6A), setting the scanning zones (FIGS. 6B-6D), setting the acquisition strategy (FIG. 6E) and schematically depicts scanning with multiple planes (FIG. 6F) or with a 3D spiral (FIG. 6G) around the R vector.

FIGS. 7A-7E are diagrammatic depictions illustrating the principle of generating a LineScan with the RoboScanner for a Volume Modality based on the voxel width (FIGS. 7A, 7C), the voxel depth (FIG. 7B), SP profiles of associated voxels (FIG. 7D), and an alternate AA array (FIG. 7E).

FIGS. 8A-8C depict SP profiles for Volume Modalities of the detection area of a LIF sensor (FIG. 8A), the corresponding profiles on a Log Scale graph (FIG. 8B) and the B1-profile of a circular RF coil (FIG. 8C).

FIG. 9 is a diagrammatic description depicting generation of a LineScan with the RoboScanner for a tomographic modality.

FIG. 10 is a flowchart of the processes and interfacing from planning to the generation of the LineScan.

FIGS. 11A-11D illustrates different scanning patterns with the Limited-FOV modalities of the system.

FIGS. 12A-12C depict examples of optical probe scanning protocol showing the streaming optical data collection (FIG. 12A), streaming-like collection of OCT interleaved with MRS and timing diagram of the DOF (FIG. 12B) and (FIG. 12C). FIGS. 13A-13B depict holders for excised tissue for ex-vivo studies.

FIG. 14A-14C illustrate scenarios and information flow for lesion detection (DETECT) and characterization (CHAR), with MRI, OCT+MRS and tissue biopsy for current MRI & MRI guided biopsy (FIG. 14A), MRI & OCT+MRS (FIG. 14B), and OCT+MRS ( ).

FIGS. 15A-15B are example implementations of the system for a device that combines MRI as the Wide-FOV modality, and LIF and OCT as the Limited-FOV modalities for breast cancer scanning.

FIGS. 16A-16D show designs for combining LIF and NMR and CoM and NMR as side-looking Laser/Light-induced fluorescence (LIF) plus MR dual sensor (FIG. 16A), a forward-looking Confocal Microscopy plus MR dual sensor (FIG. 16B), 3D simulation of isosurface dual LIF+MR modality scanning (FIG. 16C), and streaming-like collection of LIF interleaved to MRS (FIG. 16D).

FIG. 17 is a timing diagram and triggering scheme for LIF/CoM (OPT) and MRS data collection.

FIG. 18 is a flowchart illustrating a method for performing multimodality and multilevel scan for tumors with one or more paths of scanning.

FIG. 19 is a flowchart for using the device for the local infusion of contrast agent that may or may not require activation. The device may also allow the removal of excess contrast agent

FIGS. 20A-20E illustrate the process implemented for generation of LineScans with the delivery device (FIG. 20A) and the images so produced via MR (FIG. 20B), GRE MRI (FIG. 20C) and GRE (FIGS. 20D-20E).

FIGS. 21A-21E depict the architecture (FIG. 21A) and the physical prototypes of the device (FIG. 21B), the Limited-FOV sensor of the device (FIG. 21C), the optical encoders (FIG. 21D) and the optical end stop-switches (FIG. 21E).

FIGS. 22A-22D illustrate results from a prototype probe with a miniature RF coil as the Limited-FOV sensor utilizing a dual compartment phantom with gelatin and oil for a stacked single-Pulse Spectra full bandwidth in 3D (FIG. 22A), a graph of integrated intensity vs. Z for 5 peaks (FIGS. 22B-22C) and pseudo-color map of the integrated intensities of bands at certain spectral densities (FIG. 22D).

### DETAILED DESCRIPTION OF THE INVENTION

As used herein, the following terms and phrases shall have the meanings set forth below. Unless defined otherwise, all technical and scientific terms used herein have the same meaning as commonly understood to one of ordinary skill in the art.

As used herein, the term, “a” or “an” may mean one or more. As used herein in the claim(s), when used in conjunction with the word “comprising”, the words “a” or “an” may mean one or more than one. As used herein “another” or “other” may mean at least a second or more of the same or different claim element or components thereof. The terms “comprise” and “comprising” are used in the inclusive, open sense, meaning that additional elements may be included.

As used herein, the term “or” in the claims refers to “and/or” unless explicitly indicated to refer to alternatives only or the alternatives are mutually exclusive, although the disclosure supports a definition that refers to only alternatives and “and/or”.

As used herein, the term “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value, e.g., having the same function or result. In some instances, the term “about” may include numerical values that are rounded to the nearest significant figure.

As used herein, the term “subject” refers to any recipient of in situ, ex vivo or in vivo multimodal scanning, imaging and/or biosensing as described herein.

In one embodiment of the invention there is provided an automated system for multimodality imaging of a tissue in a subject, comprising a robotic delivery device configured for mechanically scanning the area of interest; a computational core comprising a plurality of software modules electronically interlinked with the delivery device; one or more interfaces between one or both of the computational core, the delivery device and an operator thereof; at least one limited field of view sensor modality mechanically linked to or carried on the robotic delivery device and electronically linked to the computational core; and at least one wide field of view imaging modality electronically linked to the computational core.

In this embodiment the robotic delivery device may comprise one or more probes configured to link one or more limited field of view sensor modalities to the area of interest, means for dimensionally translating the one or more sensors along one or more axes of the delivery device and an acquisition unit for acquiring sensor data during translation thereof. Further to this embodiment the robotic delivery device may comprise a medium for coupling the one or more sensor modalities to the tissue and/or means for image-guided tissue sampling mechanically linked to or carried on the robotic delivery device and electronically linked to the computational core. In another further embodiment the robotic delivery device may comprise one or more channels configured to accommodate means for delivery associated with a type of the device, to deliver locally one or more therapeutic agents, one or more diagnostic agents, a combination thereof or to locally deliver one or more contrast agents compatible with the sensor modality or the imaging modality, or remove one or more fluids obstructing the link between the sensor modality and the tissue.

In these further embodiments the robotic delivery device may be a needle, a catheter or an add-on component to another interventional or surgical device. Also, the contrast agent may be activatable and/or targetable. Particularly, if the contrast agent is activatable, the channel associated therewith is electronically linked to a means for activating the contrast agent disposed on the delivery device or external thereto. In addition, the limited field of view sensor modality comprises one or more of optical coherence tomography, light induced fluorescence, confocal microscopy, high-resolution ultrasound, or MR spectroscopy or imaging with miniature radiofrequency coils tuned to appropriate nuclei. The wide field of view imaging modality may comprise magnetic resonance imaging (MRI), non-digital or digital x-ray, mammography, computer tomography (CT), and 2D or 3D ultrasound.

Also in this embodiment the plurality of software modules comprise in electronic communication a planning module configured for planning a collecting of multimodal data, a processing module configured for analyzing the collected data, a tissue sampling module configured for sampling at one or more sites of interest on or in the tissue, a visualization module configured for graphically outputting the analyzed data, a fusion module configured for co-registering and co-visualizing of the one or more sensor modalities and the one or more imaging modalities, and a control module configured for linking the delivery device to the computational core.

In one aspect the planning module is configured to execute tasks to process the one or more imaging modalities, to determine scanning zones for the one or more sensor modalities based on the one or more processed imaging modalities, to determine a trajectory vector R for the scanning zones, and to implement a sensor modality data acquisition. In another aspect the processing module is configured to execute tasks to order the data collected from the one or more sensing modalities, to process the ordered data and to implement analysis of multimodal data. In yet another aspect, the tissue-sampling module is configured to execute tasks to determine one or more signal values received at one or more positions in one or more scanning zones during scanning of the sensor modality and to enable sampling or biopsy of the tissue at the one or more positions based on the signal values. In yet another aspect the fusion module is configured to execute tasks to generate voxels or tissue volumes from data collected from the one or more sensor and imaging modalities, to produce spatial maps of the collected data and to present the mapped spatial data to the visualization module. In yet another aspect the control module is configured to execute tasks to control scanning, to actuate the collection of data with the wide field of view imaging modality and the limited field of view sensor modality according to the collection plan, to actuate tissue sampling and manage the tissue samples, and to deliver one or more of a contrast agent, therapeutic agent or diagnostic agent. Particularly in this aspect, if the contrast agent is activatable, the control module is further configured to execute the task to engage means for activation of the contrast agent.

In another embodiment of the invention there is provided a method for multimodal image scanning of a tissue, comprising the steps of selecting at least two modalities and co-registering the same; selecting an area of interest on or in the tissue; scanning the area of interest via the co-registered modalities; and analyzing multimodal data collected during scanning.

Further to this embodiment the method may comprise obtaining a tissue sample at the area of interest for analysis. In another further embodiment, the method may comprise delivering locally one or more of a therapeutic, diagnostic or contrast agent prior to or during scanning. In yet another further embodiment, the method may comprise visually displaying the analyzed data as one or more spatial maps. In yet another further embodiment the method may comprise diagnosing a pathophysiological condition based on the analyzed multimodal data. An example of a pathophysiological condition is a cancer.

In one aspect of all embodiments, prior to the scanning step the method comprises selecting at least one imaging modality as the two or more modalities; determining one or more zones for scanning based on the at least one selected imaging modality; determining a trajectory vector R for each of the at least one scanning zones; selecting at least one sensor modality as the two or more modalities; and implementing acquisition of sensor modality data. In another aspect, the step of analyzing the multimodal data comprises generating voxels or tissue volumes from the multimodal data; and producing spatial maps of the collected data representing each scanned position comprising the one or more scanning zones.

In certain embodiments the at least two modalities comprise at least one wide field of view imaging modality and at least one limited field of view sensor modality. Examples of these modalities are as described supra. Also in all embodiments the tissue may be imaged in situ, in vivo or ex vivo. In addition, imaging the tissue comprises imaging a tumor or imaging a biochemical comprising the tissue.

In yet another embodiment of the invention there is provided a multimodal tissue scanning system, comprising a robotic delivery device configured to deliver a scanning signal from at least one sensor modality to one or more scanning zones on or in the tissue and to collect data during scanning, where the one or more scanning zones are determined from one or more selected imaging modalities; and where the robotic delivery device is electronically linked to a planning module configured to plan collection of multimodal data during scanning; a processing module configured to analyze the collected data; a visualization module configured to graphically output the analyzed data; a fusion module configured to co-register all the modalities and co-visualize the analyzed scanning data; and a control module configured to link the delivery device to the modules.

Further to this embodiment the multimodal tissue scanning system comprises means for image-guided tissue sampling mechanically linked to or carried on the robotic delivery device and electronically linked to a tissue sampling module configured to enable sampling or biopsy of the tissue at the one or more scanning zones. In another further embodiment the multimodal tissue scanning system comprises medium for coupling the scanning signal to the tissue; a channel to deliver locally to the tissue one or more therapeutic agents, one or more diagnostic agents or a combination thereof; or a channel to locally deliver to the tissue one or more contrast agents compatible with the one or more modalities.

In both embodiments the system may be configured to dimensionally translate the one or more sensor modalities along one or more axes of the delivery device. Also, the system may be configured to image tissue in situ, in vivo or ex vivo. Furthermore, the one or more sensor and imaging modalities are as described supra.

Provided herein are methods, devices and systems for performing multimodality imaging and/or biosensing that incorporate one or more modalities or sensors that have limited tissue penetration, herein referred to as Limited Field-of-View or equally Limited-FOV modalities or sensors, and another modality that does not have a particular field-of-view limitation, herein referred to as Wide-FOV modality. Multimodal imaging as described herein is useful for macroscopic or tissue level imaging of the area of interest, and in particular for guiding the scanning of the Limited-FOV modality. The system may utilize at least one Wide-FOV imaging modality and at least one imaging and/or spectroscopy and/or non-imaging Limited-FOV modality or biosensing. Moreover, the image-guided scanning described herein is performed by means of an actuated or robotic device that mechanically scans the area of interest by carrying one or more of the Limited-FOV sensors. The actuated or robotic device is referred to herein as a RoboScanner and the image-guided scanning is referred to as RoboScan or RoboScanning.

The methods, devices and systems described herein are enabling technologies designed to combine with current or future imaging systems and/or modalities and/or sensors to facilitate the placement and scanning using sensors with limited tissue penetration. Particularly, the system is useful for performing multimodality imaging and/or spectroscopy of tissue in situ, in vivo or ex vivo in a subject. The system may be adapted to or configured for in situ therapy planning, therapy monitoring by multi-modality and multi-level characterization of tissue. As a non-limiting example, the microscopic RF antenna may be utilized to access the content of a biochemical or other element, such as choline in breast tumors, or image and characterize other tumors or cancers, such as, but not limited to, prostate cancer. The system may perform in vivo experimental studies on animal models, for example, but not limited to, the experimental study of diseases, pharmaceutical agents, therapy methods, etc. It is contemplated that such applications may require an appropriate frame for combining the delivery device described herein and the animal. The system may perform ex vivo studies of tissue specimens for clinical use, i.e. characterization of patient specimens, or experimental use. As with in vivo experimental studies, ex vivo studies may require an appropriate frame and sample holders for combining the delivery device and the specimen.

Moreover, the system can be adapted to further guide and refine biopsy procedures with third-party biopsy systems and in situ ablation of tissue, e.g., tumors. In addition, the system may function as a generic platform for carrying any sensor. Furthermore, the system may be utilized as a research and development tool for biosensors.

### I. RoboScanner System Components

### Delivery Device

A delivery device with actuators and position encoders may be computer- or manually-controlled or can alternate operation between computer- and manually-control, depending on the specific use. The delivery device is configured to mechanically couple all modalities, both Wide- and Limited-FOV, for their co-registration, fusion and cross-modality interpretation for the diagnosis of lesions in situ, from modalities that may probe the tissue at different levels. The delivery device is configured to mechanically co-register all modalities at the same spatial coordinate system, based on the known position and/or controlled motion of the delivery device relative to the Wide-FOV modality and the known position and/or controlled motion of the different “Limited-FOV” modality sensors relative to the thereto.

Particularly, the delivery device is an actuated implement/device, i.e. a robotic device, that mechanically scans the area of the tissue of interest by carrying one or multiple Limited-FOV sensors or any sensor of limited tissue penetration. This mechanical implement addresses and facilitates several aspects of the multimodality imaging/spectroscopy, including, but not limited to:
a) addressing the limited tissue penetration of the Limited-FOV modalities, that is critical for their in situ invention, by locally deploying the Limited-FOV sensors inside or otherwise at the vicinity of the tissue that can be interrogated,
b) spatially scanning with the Limited-FOV sensor(s) the tissue of interest, thereby: i) assessing local inhomogeneities of the morphological and biochemical properties of tissue, ii) generating “Line scans” along the axis of the probe that can be used to produce one-dimensional maps of the morphologic or biochemical properties of tissue that in turn can be used for comparison purposes, e.g. normal vs. tumor, and (iii) allowing user-defined or automated assignment of different acquisition parameters or scanning strategies based on the particular needs of the diagnosis, e.g. high spatially density of data collection at the boundaries of tumor and healthy tissue and
c) inherently co-registering multimodal data based on mechanically coupling of all modalities, thereby allowing for information fusion and cross-modal interpretation of the raw data and their maturing to diagnostic decision making information. In particular in the case when the Wide-FOV modality is MRI, then with the described approach all modalities are co-registered to the inherent coordinate system of the MR imager.

The delivery device incorporates an appropriate mechanical implement or stage or base that carries one or more Limited-FOV sensors at well defined positions relative to a known point. The sensors may preferentially be carried inside the hollow part of the delivery device or externally to it. Moreover, the “delivery device” has appropriate access windows on its body allowing access of the Limited-FOV sensors to the tissue, for example, a quartz window for optical sensors, such as OCT or LIF. An additional mechanism may be embedded into the delivery device for extending and retracting a Limited-FOV sensor beyond its distal end as means of exposing the sensor to the tissue for data collection, such as when utilizing a miniature RF coil for the collection of MR spectra. Furthermore, a suction implement may be incorporated such that a small portion of the tissue inside the hollow portion of the delivery device to improve proximity of the Limited-FOV sensors to the tissue and/or facilitate fine-needle aspiration tissue biopsy.

Generally, the delivery device can be adjusted to accommodate and scan via the methods disclosed herein with any optical imaging and/or spectroscopy method that operates with endogenous or exogenous agents. Thus, the delivery device can accommodate sensors of different modalities. The delivery device is designed for the appropriate accommodation into the hollow channel of the delivery device of all links needed for the operation of the Limited-FOV sensors, for example, but not limited to, wires, coaxial cables, optical fibers, etc. The hollow portion of the delivery device also may be filled with appropriate material or medium, for example, saline or a fluid with appropriate optical properties, for the better operation of the Limited-FOV sensors, i.e. for better coupling of the sensor to the interrogated tissue.

The delivery device may be a needle, a catheter, or an add-on component to an existing, e.g. third-party OEM, interventional or surgical device The delivery device may incorporate one or more of at least one channel for fine needle aspiration (FNA), at least one channel for locally delivering therapeutic agents or at least one channel for locally delivering contrast agents utilizable with the Limited-FOV or the Wide-FOV modality to further enhance contrast and thus the detection and characterization of a pathologic site. Those agents may be, but are not limited to, “smart” agents that target genes, receptors etc. Such agents may be activated as example, but not limited to, the presence of biochemical entities, such as enzymes, that are characteristic of pathology of interest. The critical aspect of utilizing contrast agents is that the contrast agent is preferentially activated and generates a signal that is detectable by the sensors when it interacts or binds to appropriate biochemical entity. This ensures that the sensors are not saturated by the presence of high quantities or concentrations of contrast agent at their vicinity. A clear benefit of localized delivery of contrast agent, as compared to a systemic one, is the far less dose needed; this reduces the cost of the procedure and the potential side effects, e.g. toxicity, to the patient.

The targeted diagnostic and therapeutic agents may be utilized in the system for combined diagnosis and therapy similarly to image-guided tissue sampling. Specifically, as the device scans, depending on the findings from the sensors, the operator may activate the local delivery of a therapeutic agent. Moreover, this procedure can be automated or semi-automated using appropriate algorithms that use criteria for the local release of therapeutic contrast agent either after approval by the operator who becomes aware of this particular finding, i.e., semi-automated or in a fully automated scheme.

The delivery device may incorporate thereon or therein position encoders for determining the length, direction and speed of the motion of the Limited-FOV sensors. Position encoders may be, but are not limited to, linear and/or rotational optical differential encoders. The delivery device may preferentially incorporate optically actuated stop switches to ensure the operation of the scanning portion of the “delivery device” between two positions for safety and kinematic purposes.

The delivery device enables translation of Limited-FOV sensors along its axis for scanning the tissue, thereby generating a one-dimensional scan of the corresponding physical properties of tissue that can be assessed by the particular sensor. This 1-dimensional scan is preferentially herein referred to as Line-Scan or LineScans. Moreover, this translation may occur inside the hollow part of the delivery device, and may be performed manually by the operator, may be actuated by means of actuators, such as motors, pneumatically or hydraulically, and may be controlled by computer software and/or by the operator using manual switches that activate those actuators.

Probes of the delivery device may have appropriate access windows for the Limited-FOV sensors to access the tissue. This access window is covered an appropriate material, e.g. glass, quartz, etc, selected for allowing the appropriate electromagnetic spectrum or ultrasound waves that operate the sensors to pass therethrough. The access window may be an elongated slit along part of or along the entire length of the delivery cannula that allows scanning of the sensors by their mechanical translation inside and along the long axis of the cannula, or may be a small opening, in which case scanning is performed by moving the entire cannula. In the latter case it may be further desirable that the cannula is pulled. Pulling the cannula may be farther suitable for when the probe is for confocal microscopy (CoM).

### Limited-FOV Sensors

A plurality of sensors for the Limited-FOV modalities mechanically attached onto and/or coupled to the delivery device. Limited-FOV sensing includes any type of sensing, such as biochemical or physiologic, required for collection of needed information. Limited-FOV sensing can be implemented by means of localized detection of the entities that produce the signal that is detectable by a sensor that is locally placed within the delivery device or can be connected to the site to collect the sensing signal, such as, but not limited to, optical fibers or other type of antennas. Herein, any type of Limited-FOV sensing in general is referred to as Limited-FOV modality. Limited-FOV modality(-ies) are utilized to scan and to collect biologic- and/or patho- or normal-physiologic-relevant information at a localized areas of the tissue of interest and may contribute to the interpretation of the Wide-FOV modality.

Limited-FOV sensor modalities include those for optical imaging and/or optical spectroscopy currently available or that maybe developed in the future that are of limited tissue penetration and require the localized presence of the sensor to alleviate the limited tissue penetration. Limited-FOV modalities are any molecular or cellular level modalities such as, but not limited to, optical coherence tomography (OCT), light induced fluorescence (LIF), confocal micropscopy (CoM) high resolution ultrasound, and MR spectroscopy (MRS) or MR imaging (MRI) with miniature radiofrequency (RF) coils tuned to the appropriate nuclei depending on the sought biochemical information. Moreover, the Limited-FOV modalities may comprise a lab-on-a-chip device.

### Wide-FOV Imaging Modality

At least one Wide-FOV imaging modality to image a large area of tissue in vivo or ex vivo, or a sample of material to i) identify a lesion, ii) potentially characterize the tissue, iii) guide the placement of the delivery device or devices that carry and manipulate the sensor(s) of the Limited-FOV modality(-ies), iv) select the mode of scanning of the Limited-FOV modalities, and v) provide information for multi-modal based interpretation of all data for Limited- and Wide-FOV modalities. Wide-FOV modalities are, but not limited to, magnetic resonance imaging (MRI), non-digital or digital x-ray, including specialized imagers such as mammography, computer tomography (CT), and 2D- or 3D-ultrasound. Examples of Wide-FOV modalities are, but not limited to, optical coherence tomography (OCT), light induced fluorescence (LIF), high resolution ultrasound (US), MR spectroscopy (MRS) or high resolution MRI with miniature RF coils.

### Software, Hardware and Interfaces

Generally, as is known in the art, computers, personal computers and other computer devices and computer systems are networkable and comprise memories and processors effective to receive and store data and execute instructions to process data received from the Roboscanner System, monitors, data delivery and processing hardware and software, etc. and may be wired or wireless, hand-held or table top utilizable.

Software modules for, but not limited to, planning the scanning strategy, i.e. use the “Wide-FOV modality” to determine the patterns of scanning for the Limited-FOV, controlling the actuators of the robotic device and performing cross-modal analysis and generation of information-rich, e.g. anatomical and biochemical features, spatial maps of the tissue. The system further may incorporate expert, i.e., machine-learning and or data-mining based, algorithms and software for automated or assistive interpretation of multi-modal data. Moreover, the expert system may include a visualization/graphics output, for example, to a monitor screen of any type, for presenting the results to the operator. The software may enable signal control and co-registration of Limited-FOV and Wide-FOV modalities. In addition, software may incorporate error compensation in the control signals and/or in the co-registration software routine to address non-linearities in the spatial encoding of the Limited-FOV modalities.

The system comprises a human-machine interface to the operator. The system further has interfaces, e.g., cables, optical cables, co-axial cables, etc. to the Wide- and Limited-FOV scanners, electronics, and computer. The system may comprise a PC or dedicated PC board or an embedded PC system to run the software modules. The computational core may reside on a dedicated embedded computing unit, a card, such as a PCI or PCMCIA card, a microprocessor unit and can be a compact hardware piece that can be an add-on component to a desktop or portable computer, including but not limited to a Personal Computer (PC).

The system can operate as a stand-alone unit or as an add-on to a third party original equipment manufacturer (OEM) imaging and diagnosis equipment or products. Examples of such third party products may be, but not limited to, biopsy systems, vacuum assisted devices, biopsy guns, etc. Moreover, Limited-FOV sensor(s) maybe designed and/or constructed by a third party OEM. The system, as a whole or parts thereof, is physically constructed to be compatible with the particular needs of other modalities, for example, MRI, CT, or US compatible. This can be achieved via methods known to those skilled in this art by the appropriate choice of construction material, preparation of different components, such as electrically shielding the cables, software for filtering or compensating for artifactual effects, etc.

### Multi-Modality Fusion Module

The system software comprises a fusion module for performing the co-registration and co-visualization, i.e., fusion, of the different modalities, including the Wide-FOV and the Limited-FOV modalities. The fusion module has, as input, the spatial sensitive area of signal reception for all the employed Limited-FOV sensors. The sensitivity profile may be expressed as a mathematical entity of the type SP(J, P, x, y, z), where J is the index of the specific Limited-FOV sensor, P are the specific parameters of the sensor, (x, y, z) are the coordinates of the position in space, and SP is the detection sensitivity of the sensor that is expressed by any means appropriate for the modality and as is known in the art.

The SP(J, P, x, y, z) mathematical entity may be stored in the system's software may include a data-base of library of pre-determined sensitive area of detection of the Limited-FOV sensor(s). In addition, or alternatively, the system may incorporate appropriate methods for determining the sensitive area of detection of the Limited-FOV sensor(s) experimentally in situ and on-the-fly during a diagnostic procedure. In particular, the system's software may include a software module for determining the sensitive area of an RF coil for MR localized biosensing. This module is not limited to, but can be based on the Biot-Savart Law. Also particularly, the system's software may include a software module for determining the sensitive area of detection of an optical sensor.

The system's software includes a software module that orders and places the raw and processed data collected with the different Limited-FOV modalities on the spatial positions that they were collected. This software module further incorporates the sensitivity profile (SA) of detection of the Limited-FOV sensor(s). The fusion module executes a series of tasks including, generating voxels, i.e. tissue volumes from which a particular set of data were collected and producing spatial maps of the data collected with the Limited-FOV modalities for use in clinical diagnosis or analysis of experimental data. Non-limiting examples of such maps are the spatial distribution of metabolites, such as choline, in breast tumors measured with a miniature RF coil, or endogenous or exogenous fluorophores measured with a LIF sensor. The system's software may include a software module for basic processing and loading the multi-modal data and presenting them to a visualization window.

### II. Scanning

The RoboScanner system comprises a process of tasks for planning, collecting and co-visualizing multimodal information. The selection of the scanning zone may be performed manually or may be semi-automated or automated by the software described herein.

### Image Guided Tissue Sampling

The sensor may be used to guide the localized physical sampling of tissue, i.e. biopsy, for example by means of a fine-needle-aspiration (FNA)-like subassembly or add-on to the scanning device. The tissue sampling subassembly collects multiple samples guided by the multimodality data. Such localized physical sampling of tissue is based on two aspects particular to this intervention the high spatial resolution achieved by the high resolution mechanical scanning of the sensors, and the high specificity of the collected information about the type of tissue that is interrogated. This is due to the inherent particular tissue contrast or biochemical content properties, based on endogenous biochemical entities, and/or the use of suitable exogenous contrast agents delivered systemically, e.g. by means of an intravenous injection or locally by means of a suitable dedicated agent delivery channel, as described herein.

Tissue sampling, for example, but not limited to, fine needle aspiration may be performed in multiple sites along the path of scanning with the sensors. Such multi-site sampling maybe desired to, inter alia, compare healthy to diseased tissue to better identify differences pertinent to diagnosis, to identify the margins of healthy and diseased tissue, to identify inhomogeneities inside the healthy or diseased tissue. When multiple sites are physically sampled or biopsied, e.g. with FNA, then the tissue samples are individually stored and indexed to correlate to the particular signals collected at this site.

Image-guided physical sampling of tissue may be performed manually or may be semi- or fully-automated. In the manual mode of operation, the expert operator is on-line, selects the site and activates the sampling mechanism. The operator is tasked to visualize the data as they are collected by the scanning apparatus at a particular site S_{J}, where J is the index of scan along the path of scanning, based on this information, and criteria known to those skilled in the art, to decide whether a physical sample must be collected at this particular site S_{J} and to actuate the sampling mechanism to perform physical sampling, for example, by clicking on a button on the control GUI, or a switch on a joystick or other similar means. This action further activates the mechanism for acquiring the physical sample and creates the appropriate entry to correlate this specific physical sample to the sensor data at this site.

Alternatively, in semi-automated or fully automated multiple physical tissue sampling, a degree of automation of the procedure can be achieved at the level of selection of the site S_{J} for physical tissue sampling, based on a software module comprising algorithm(s)). This software module accesses the raw or processed data from the sensors, further processes them and, based on certain selection criteria, actuates the tissue sampling subassembly. Examples of criteria for the automated software based actuation of sampling may be based preferentially on, but not limited to, signal properties related to endogenous or exogenous agents. This may include a threshold or a combination of thresholds for one or more values of the signals, the appearance of one or more signals, for example, choline in NMR, that are characteristic of a particular type of tissue or pathologic condition of tissue, a more intelligent algorithm, for example one that uses Boolean operations to combine the changes in multiple signals, and a simple schedule of sampling, as example every certain step in the spatial advancement of the sensors with the mechanical positioned, e.g. every 1 mm.

The device that combines a tissue sampling subassembly is designed and constructed in such a way the sampled tissue spatially coincides with the area probed by the sensors, i.e. S_{J}. This ensures the direct spatial correlation of the sampled tissue and the signals from the sensors, i.e. the tissue sampled is the exact tissue that has been or is interrogated by the sensors. Thereby, the sampling mechanism can access tissue and sample it forward of the probe at its distal tip, if the sensors are forward looking, on its side, that is, orthogonal to its axis and at a location not at the distal tip of the probe, if the sensors are side-firing or at an angle relative to the tip of the axis of the probe at an angle. Examples of the first type are when the sensor is a forward looking confocal microscope or OCT or LIF. Examples of the second type is when the sensor is a side-looking OCT or LIF. Many different examples and cases can be implemented by the specialists in the field that satisfy the requirement that the sampled tissue is the same with the one interrogated by the sensors.

### Limited-FOV Scanning

The delivery device has an appropriate number and type of actuated Degrees of Freedom (DoF) for spatially positioning and scanning with the Limited-FOV sensor(s). These DoF may be, but are not limited to at least one translational DoF along the axis of the delivery device, which is used for the one-dimensional (1D) scanning with the multi-sensor to generate the LineScan according to the scanning pattern. Controlled linear movement of the Limited-FOV sensors generates one or more 1D spatial distributions (LineScan). This translational DoF(s) is used further for 1D scanning of more than one zones in the tissue with the multi-sensor, based on the “scanning pattern”. The translational DoF(s) is further used for positioning the multi-sensor at a specific linear (including initial) position.

Alternatively, DoF may be at least one rotational DoF around the axis of the delivery device. The rotational DoF(s) is/are used to continuously and radially scan the tissue with the multi-sensor around the axis of the delivery device. Controlled rotational movement of the Limited-FOV sensors generates one or more 2D spatial distributions (Planar Scans). The rotational DoF(s) is used further to radially scan one or more arc areas of the tissue with the multi-sensor. The rotational DoF(s) is used further to radially position the multi-sensor at specific angular, including an initial, position.

The scanning of each zone, selected at the Scanning Zones task, with an acquisition strategy that determines the exact way the Limited-FOV sensor data are spatially collected. Different patterns of scanning can be implemented depending of the DoF of the delivery device, such as, but not limited to, multiple planes around the R vector trajectory or a 3D spiral centered on the R-vector trajectory. The operator determines how the zones selected with the acquisition strategy are scanned to better assess the spatial distribution of the interesting moieties. This defines the specific way Limited-FOV sensing can be implemented, i.e. Scanning Modes. Specifically, the Scanning Mode may be, but not limited to, the following:
i) Scanning and collection of data with the Limited-FOV sensors using the delivery device along a trajectory can be performed in multiple distinct steps with the sequence: (move from position A to position B without collection of data during motion)-(stop)-(collect data)-(repeat until completing scanning);
ii) Scanning and collection of data with the Limited-FOV sensors using the delivery device along a trajectory can be performed in multiple distinct steps with the sequence: (move from position A to position B and collect data of, e.g., modality M1)-(stop)-(collect data of, e.g., modality M2)-(repeat until completing scanning); or
iii) Scanning and collection of data with the Limited-FOV sensors using the delivery device along a trajectory can be performed in a continuous fashion with the sequence: (continuously move between the two positions A and B, while interleaving the collection of multiple modalities, e.g., modalities M1 and M2.

The delivery device may be maneuvered and placed along the scanning trajectory R as hand-held by the operator, for example, a radiologist, surgeon, technician, or researcher, anchored onto a non-actuated frame/base for positioning relative to the patient or target or attached to another robotic device for positioning relative to the patient.

The operator may manually or automatically select a scanning strategy that may entail the combined or independent movement of the Limited-FOV sensors. As example, combination of LineScans and PlaneScans generate 3 dimensional multi-modal images (or scanning patterns). Such 3D images (or scanning patterns) can also be implemented by means of spiral scanning acquisition schemes. The multimodal Limited-FOV data that generates the LineScans may be organized and stored in a LineScan array that parallels the scanning of the delivery device.

### Mechanical Multi-Modality Co-Registration

Spatial co-registration of the different modalities may be based on the mechanical spatial link of the Limited-FOV sensors relative to the Wide-FOV modality. Fiducial markers may be implemented, for example, when the Wide-FOV modality is MRI and compartments are filled with Gd-based contrast agents. Moreover those compartments may be wrapped into an inductively coupled RF coil for further enhancing their signal.

During scanning, implementing registration comprises determining an initial position of the delivery device from the Wide-FOV images using the fiducial markers. The forward kinematics of the delivery device are continuously solved by the control software. The transient position of the multi-sensor is determined on-the-fly by the initial position and the forward kinematics and furthermore is stored in a computer file managed by the computational core.

### Generation of LineScans

Different acquisition strategies can be defined based on the specific properties of the Limited-FOV modalities and the characteristics of their sensors. Scanning strategies correspond to different patterns of actuating the Limited-FOV sensors by means of the appropriate actuators of the RoboScanner by means of control commands generated by the control module of the computation core of the system. For example, control of scanning may be implemented with, but is not limited to, a series of control pulses that are directed to the appropriate motor controller from the control module in the computational core.

Provided herein are methods for collecting and organizing of the Limited-FOV data spatially encoded to the Wide-FOV modality. As a representative example, such methods are applicable to the Limited-FOV modality optical methods spatially encoded to a Wide-FOV modality that is the MR coordinate system. Since spatial registration is achieved via a mechanical link and considering that the only possible motion with a rigid probe is a linear translation along its axis the present invention provides a method for defining an optical voxel attached to the sensing site of the optical probe, or in general of the Limited FOV modality sensor, that can scan the tissue linearly and samples optical data at discrete positions, known relative to the MR coordinate system, thereby defining a spatial dimension of the MR-registered line scan. In a related method, the optical voxels are correlated or registered with the MR coordinate system via an Acquisition Array (AA) of the optical voxels, or in general of the Limited FOV modality voxel. The AA may be a data structure that hierarchically stores optical data together with the coordinates of the position where they were recorded thereby providing a correlation functionality.

The Acquisition Array comprises a data storage structure within the Computational Core and has the following primary features:
1) A dedicated AA corresponds to each one of the Limited-FOV modality, thus, different scanning strategies can be assigned to the different modalities.
2) The AA array is composed of indexed data storage cells AA, that represents a Limited-FOV modality voxel, especially for optical methods, an optical voxel. The number of cells N (1≦J≦N) can be determined by the parameters of the acquisition protocol.
3) The index J along the spatial dimension of AA is scanned by the translation of the optical probe, or of the Limited-FOV sensor, in general, along its axis. Each cell corresponds to the specific spatial position P_{J}(x,y,z) of the J Limited-FOV data set acquisition and can be calculated from the kinematics of the probe relative to the coordinate system of the Wide-FOV modality coordinate system.
4) The informational content of each one of the AA_{J} cells depends on the modality. If it is a volume modality, for example, but not limited to, LIF and non-localized MRS, then the content of the AA cell is determined by the SP of the particular sensor. If it is a tomographic modality, for example, but not limited to, OCT, the content is a specific number of data lines, e.g. OCT lines.
5) The AA cell populated with the Limited-FOV data corresponds to a LineScan along the R vector.
6) The cells AA_{J} are virtual entities for which size is determined by the acquisition parameters, as well as by the specific type of signal they contain, i.e. the spatial distribution of Limited-FOV signal-sources. For combining and co-visualizing different Limited-FOV modalities, such as OCT and LIF, the disclosed invention utilizes the size and shape of the areas that each Limited-FOV signal is collected from, i.e. a field-of-view (FOV), the spatial relationship of those two FOV's and the exact position collected for each voxel of the different modalities. The size of the AA can be defined by the acquisition protocol and in addition, as a virtual data structure, can be processed and be re-binned to adjust the size of the AA_{J} cells, and/or can be processed by the Visualization Module to make them appropriate for co-visualization with the other Limited-FOV modalities or the Wide-FOV or all of them.

### Wide-FOV Modality: MRI

For use of an MRI Wide-FOV modality, the delivery device may incorporate one or more MRI fiducial marker(s)) at known location(s). Multiple MR fiducial markers or MR visible markers of appropriate shape may be incorporated at multiple locations to register the delivery device in 3D. The MRI fiducial marker may be based on miniature radiofrequency (RF) coil(s) which miniature coil marker(s) may be connected to the RF interface of the MR scanner via co-axial cable(s) or may operate in an inductively-coupled manner. Particularly, a fiducial marker can be the miniature RF coil used as a Limited-FOV sensor for MR spectroscopy. The MRI fiducial markers may comprise MRI contrast agent filled vials. Furthermore the robot control software enables automated control of a gradient based localized (GradLoc) technique. The software also enables autonomous control of both the MR scanner and the robot in synchronization.

Spatially co-registering modalities includes the determination or definition of a common coordinate system. When the Wide-FOV modality is MRI, the endogenous coordinate system of the MRI scanner may be used. Specifically, using MR visible markers, the initial position of the RoboScanner is determined from MR images and used to calculate the static and transient position of the sensor(s) of the Limited-FOV modalities or, in the case of an optical sensor the optically active and actuated parts of the probe, from its kinematics.

Registration of the delivery device that carries a probe and the Wide-FOV modality of MRI requires knowing the kinematics relative to the MR scanner and initial registration with On-Probe MR markers. The position where the signals of the limited FOV sensors, for example, but not limited to optical signals, are sampled, which is the position of the detecting optical heads, can be calculated from the kinematics of the probe. The kinematics are the formulas that calculate the transient position of any point of the actuating optical probe relative to an initial position, based on its specific geometry, degree-of-freedom and control signals. To determine the initial position of the probe, at the beginning of each study, it is registered to the MR scanner by determining the coordinates of the centers of MR visible markers, for example, but not limited to, cross-shaped markers. The area of the probe is imaged with a heavily T1-weighted fast gradient recalled echo for high contrast visualization of the markers (149). The coordinates of the center of the markers are extracted manually or with an image processing routine and supplied to the kinematics of the control.

Collecting morphologic and functional information may be collected from an organ-level Wide-FOV modality, i.e. MRI, with morphologic information with a Limited-FOV modality at the cellular or near-cellular level, i.e. OCT, and biochemical assaying with another Limited-FOV modality, i.e. MRS. It is contemplated that multimodal and multi-level sensing may eventually offer high sensitivity from MRI and specificity from MRS and OCT in the characterization of lesions in situ.

III. Imaging or Spectroscopy with Specific Sensors

### Limited-FOV Dual Sensor

In a particular example, a Limited-FOV sensor may be an OCT probe combined with a LIF probe, herein termed “CT/LIF dual-sensor. The OCT/LIF dual-sensor” can be implemented in different designs of optical probes that share, but are not limited to, the following general design features:
i) The detection area of the LIF spectroscopic method is centered at the tomographic FOV (thus the detection head OH-LIF_{DET} is at the same axial position with the tomography head OH-OCT). Both OH-LIF_{DET} and OH-OCT are actuated together (say with a common DoF-2) to scan along the P vector (or equally the AA array).
ii) The light source optical head (OS-LIF_{EMIT}) could be axially translatable (say with the DOF-3) impendent of the detection sub-assembly (i.e. OH-LIF_{DET} and OH-OCT) to comply with data acquisition practices with different geometry probes, as in diffuse optical imaging and LIF (150-152).
iii) To scan a different axial view (plane) of the tissue, the probe can have a rotational degree of freedom (say DOF-4) that rotates all optical source and detection elements maintaining their alignment.
iv) For practical reasons, an additional DOF-1 can be incorporated for the insertion of the probe at the location of interest before optical scanning starts. An actual probe based on the design described above can be physically prototyped and tested.

The plurality of DOF on the proposed probe indicates that there can be a large number of potential scanning paths to optically image a tissue of interest. The number of paths can be even higher considering that the number of LIF (N_{LIF}) and OCT lines (N_{OCT}) collected can be different. Since the duration of scan cannot be infinite, such as for clinical studies, and considering the limited duration of signal enhancement for some applications, it is preferable to be able to optimize protocols.

A dedicated software module can preferentially be incorporated into the computational core of the RoboScanner system for generating optimized acquisition protocols to control the scanning with the OCT/LIF dual-sensor. This software module enables algorithms that have as parameters 1) the duration of a single line of data acquisition for OCT (T_{OCT}) and LIF (T_{LIF}), including the time for electronic switching between the two modalities; 2) duration of actuation per DOF, including delays; 3) mechanical errors; 4) relative desired weighting of density of data collection (W_{DATA}) versus volume scanned (W_{VOLUME})′ and the relative weighting of number of OCT lines (N_{OCT}) and LIF scans (N_{LIF}). Scanning parameters include the number and position of OCT lines, the number and position of LIF acquisition, and the path followed by the actuated probe to acquire those points. Virtual phantoms can be generated with representations of tissues with different optical properties (153-154).

The code comprising the software module enables calculation of Forward and Reverse optimized scanning. To calculate an optimized Forward scanning protocol, for didactic purposes the algorithm simulates the covered area and the T_{ACQ} for simple intuitive scans, such as a single continuous line scan, or one that samples two distinct areas, healthy vs. lesion, as well as spirals around the axis of the probe and rectilinear scans. For Reverse scanning, the algorithm, defines the area of the lesion and, optionally, of healthy tissue for reference, which in practice are both provided by MRI, and calculates the scanning parameters for maximum density of data collected and reports the scanned tissue volume, for maximum volume of tissue scanned and reports the density of data collected and for any weighting W_{DATA} and W_{VOLUME}, for a given T_{ACQ}. Preferably, the algorithmic code may be implemented in any platform, including, but not limited to, C, C++, C#, Simulink (Mathworks), Matlab (Matworks), as are known in the art.

### Limited-FOV Radiofrequency Sensor

A miniature RF coil may be utilized as a Limited-FOV sensor modality, by itself or combined with other Limited-FOV sensors, for MR spectroscopy at lower magnetic field scanners, where the proximity of the coil substantially improves the sensitivity of the modality. Such a Limited-FOV RF sensor is dedicated to magnetic resonance imaging (MRI) and magnetic resonance spectroscopy (MRS). A biosensor for MRI/MRS can be a single or a multi-tuned radiofrequency (RF) coil. This RF coil can be tuned to the proton (¹H) resonance, and/or tuned to the phosphorous (³¹P) resonance, and/or tuned to sodium (²³Na) resonance, and/or tuned to multiple nuclei.

A Limited-FOV RF sensor can be used in one or more of both Transmission/Reception (Tx/Rx) mode, for Reception (Rx) with external RF coil(s) for RF transmission or inductively coupled for localized enhancement of the RF signal, using external RF coil(s) for reception/transmission. When the Limited-FOV RF coil is used in Tx/Rx and/or Rx mode, the coil can be interfaced via an ultra-thin co-axial cable to the RF interface of the MR scanner signal reception module. Moreover, as is known in the art, the RF coil sensor may be equipped with an RF tuning/matching circuit for tuning the coil to the desired frequency(-ies). This tuning/matching circuit can be implemented, for example, but not limited to, by locating the circuit at a distance from the RF coil or by locating the circuit in the vicinity of or onto the coil, for example, by placing capacitors onto its wire.

When utilizing a Tx/Rx or Rx RF coil as a Limited-FOV sensor, as is known in the art, means may be taken for addressing potential variable loading of the coil as the assembly of the delivery device plus the miniature Tx/Rx or Rx-only RF coil advances into the tissue, since the co-axial cable is part of the tuning circuit. This can be accomplished by additional and/or improved grounding of the co-axial, by using a miniature tuning and/or matching capacitor on the coil, i.e. at the distal tip of the delivery device or by using an inductive coupling approach.

The excitation/reception profile of the miniature RF coil may be used for determining the size of the sensitive area and the voxel that can be sensed during a scan. This can be, for example, calculated during scanning or it may be stored already in the file libraries of the computational core of the system. Those profiles can be calculated, for example, but not limited to, with the Biot-Savart Law. Particularly MRS can be performed with a single excitation pulse.

A Limited-FOV RF sensor may be utilized to perform single-voxel spectroscopy (SVS) with existing or future developed methods. Accordingly, point-resolved spectroscopy (PRESS) or Stimulated Echo Acquisition Mode (STEAM) with or without water and/or fat suppression or other type of magnetization preparation can be used according to techniques known to those skilled in this art. A Limited-FOV RF sensor with a SVS modality includes the automated, i.e., computer-controlled, relocation of the voxel of the SVS MR technique to match the position of the RF coil as the latter is scanned as described herein. This can be enabled by the actuation control software module of the delivery device that calculates the appropriate coordinates of the single-voxel in such a way that the single-voxel of the SVS technique is at a preferred position relative to the sensitive area of the RF coil on the delivery device and that supplies them to the MR scanner acquisition control software by means known to those skilled in this art. In a non-limiting example the coordinates can be sent via a dedicated Ethernet connection on-the-fly or pre-scanning in the form of an electronic file that is stored locally to the MR scanner and is loaded to the control software accessing the list of the position SVS can be performed.

As described below, the invention provides a number of advantages and uses, however such advantages and uses are not limited by such description. Embodiments of the present invention are better illustrated with reference to the Figure(s), however, such reference is not meant to limit the present invention in any fashion. The embodiments and variations described in detail herein are to be interpreted by the appended claims and equivalents thereof.

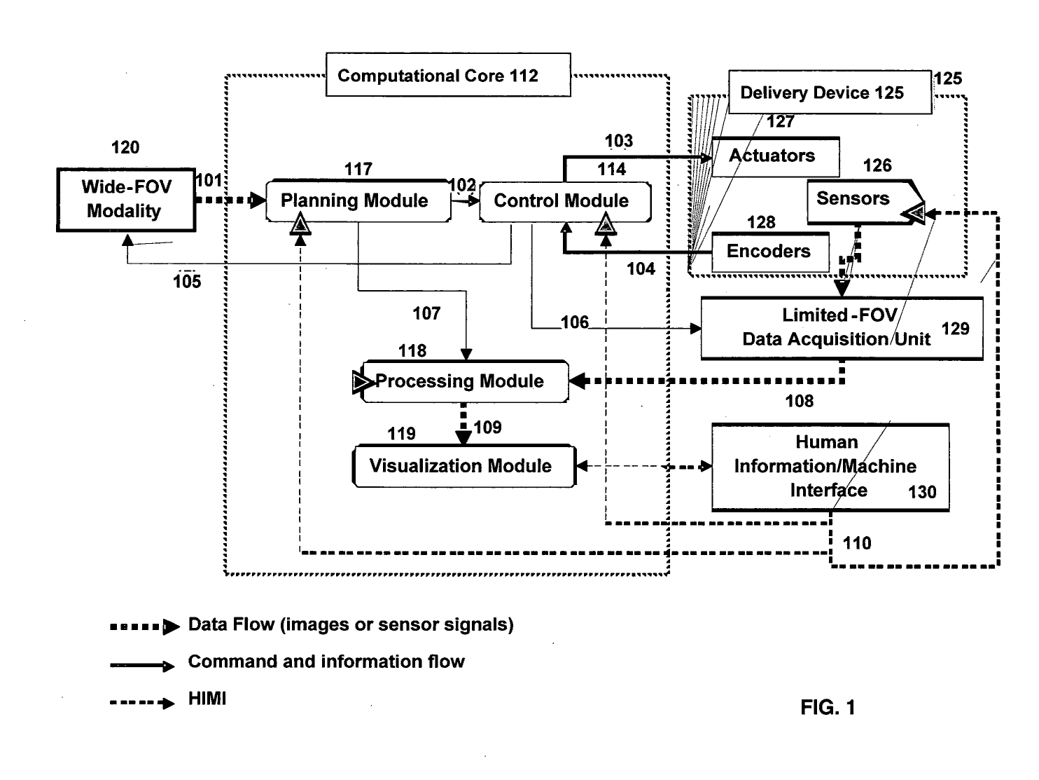

FIG. 1 is an overview of the system, processes, paths of data and control flow. The two primary elements of the system are the RoboScanner computational core 112 and the delivery device 125. The delivery device carries the sensors 126 and mechanically scans them and the computational core is composed of a multitude of software modules that perform all procedures needed for planning, scanning, processing the multimodality data, co-registering and co-visualizing them. Particularly, FIG. 1 shows, inter alia, certain data and command flow lines for Wide-FOV data at 101 from the Wide-FOV scanner modality 120, for instructions from the planning module 117 to the control module 114 at 102, for control signals from the control module to the actuators 126 of the delivery device at 103, for signals from the encoders 128 on the delivery device to the control module for closed loop feedback control at 104, for control signals from the control module to the Wide-FOV scanner 20 for triggering data acquisition and/or modifying the acquisition parameters on-the-fly at 105, for control signals from the control module to the Limited-FOV data acquisition unit 29, i.e. a signal acquisition and conditioning component included as part of the RoboScanner or of a third-party origin at 106, for controlling the data acquisition at 107, based on the results of the planning module, for Limited FOV data structure information, i.e., initial registration of the delivery device to the Wide-FOV modality 120, acquisition array and acquisition voxel, from the planning module to the processing module 118, for Limited-FOV data from the Limited-FOV data acquisition unit to the processing module at 108, for ordered and co-registered data at 109, generated from signals 107 and 108, are sent to the visualization module 119 for further processing and presentation to the operator, and for the two-way communication 110 between the human-information/machine-interface 30 (HIMI) and the computational core at 110.

With continued reference to FIG. 1, FIG. 2 depicts the computational core 112 and the control 114, tissue-sampling 116, planning 117, processing 118, and visualization 119 modules contained therein. Each one of these modules includes a plurality of synergizing routines for the different tasks that each module performs. The control module, for example, controls the actuators of the delivery device, actuates collection of data for the Wide- and Limited-FOVs, actuates tissue-sampling and management, spatial mapping of the tissue and, preferentially, the Wide-FOV scanner 120. The tissue-sampling module, which is controlled by the control module, and also may be linked to the planning and processing modules are configured to determine whether or not a tissue biopsy should be performed at any position along the scanning zones planned by the planning module based on one or more signal values received during scanning and analyzed with the processing module. The planning module is designed and implemented for planning the scanning strategy, i.e., using the Wide-FOV modality processing tools 117a and planning routines 117b, such as for trajectory R, scanning zones and acquisition strategy, for determining the patterns of scanning for the Limited-FOV. The processing module is configured to order the limited FOV data 118a, to process Limited-FOV data 118b and to conduct expert multimodal analysis 118c. The visualization module comprises visualizations routines 119a, augmented or virtual reality 119b, graphics routines 119c, and output routines 119d to perform cross-modal analysis and to generate information-rich spatial maps of the tissue, e.g. of anatomical and biochemical features thereof.

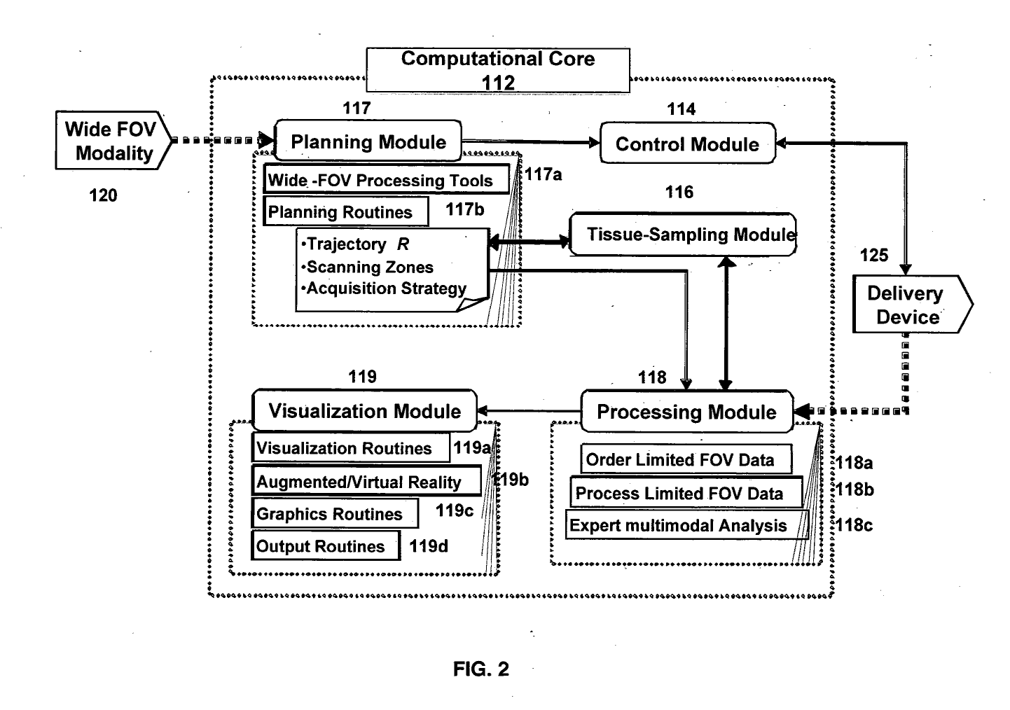

With continued reference to FIG. 1, FIG. 3 shows a representative setup of the RoboScanner hardware 300. The higher level software pieces of the computational core 112 reside in the host computer 300 while the lower level software components reside and are executed into RoboScanner unit 310. The host computer hosts and runs the higher level software pieces of the computational core. The host computer may also include peripherals 320, such as but not limited to, for data storage and printing in paper and/or film. The RoboScanner unit hosts and runs the lower level software components, particularly those that preferentially run in real-time. The RoboScanner unit can be realized in different ways such as, but not limited to, the implementations of a dedicated embedded unit 312, or an assembly 314 of data input/output (I/O) cards, for example PCI cards. The RoboScanner Unit includes digital input/output (DI/O) interfaces, digital-to-analog Converters (DAC) and other such sub-units for communication between the Host Computer and the other hardware components 330 of the Roboscanner system, for example actuators 127, encoders 128, the delivery device 125 and for receiving data from the Limited-FOV data acquisition unit 129. The RoboScanner also includes a human-information/machine-interface 340 (HIMI) for a two-way communication of the operator and the system. The HIMI includes different means of HIMI communication including, but not limited to, joystick, mouse, and optic interface. The system may also be connected to the Wide-FOV modality 120 via a LAN 350, for example, for receiving images and, if selected, for triggering and/or changing imaging parameters on-the-fly.

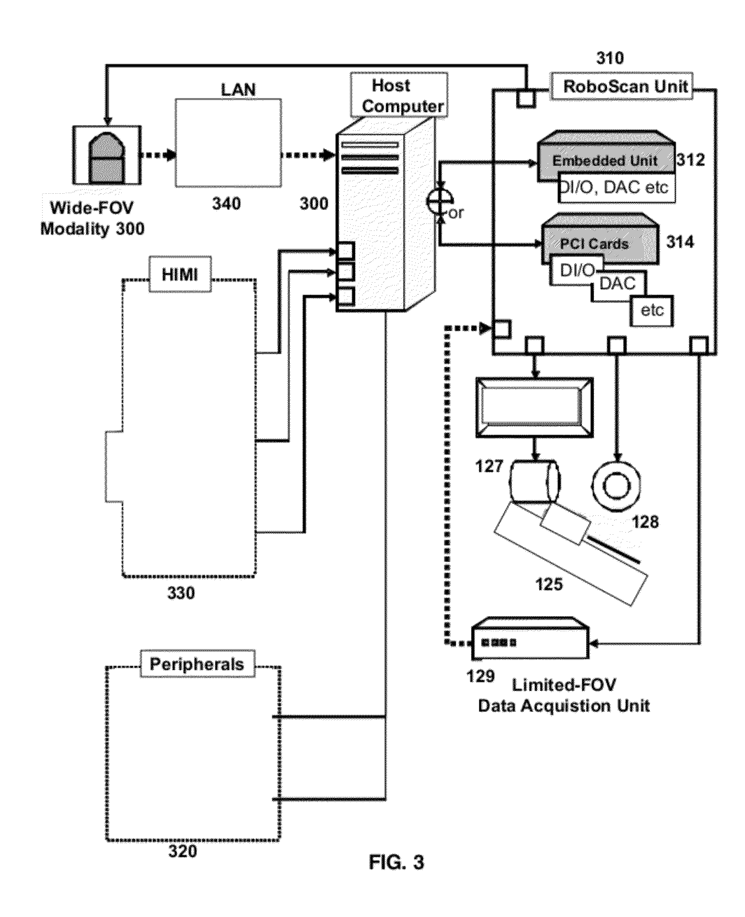

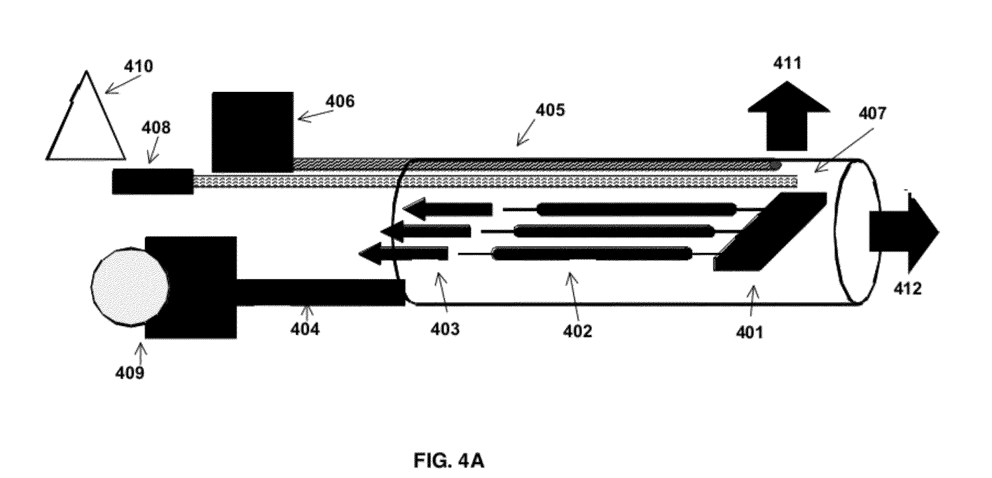

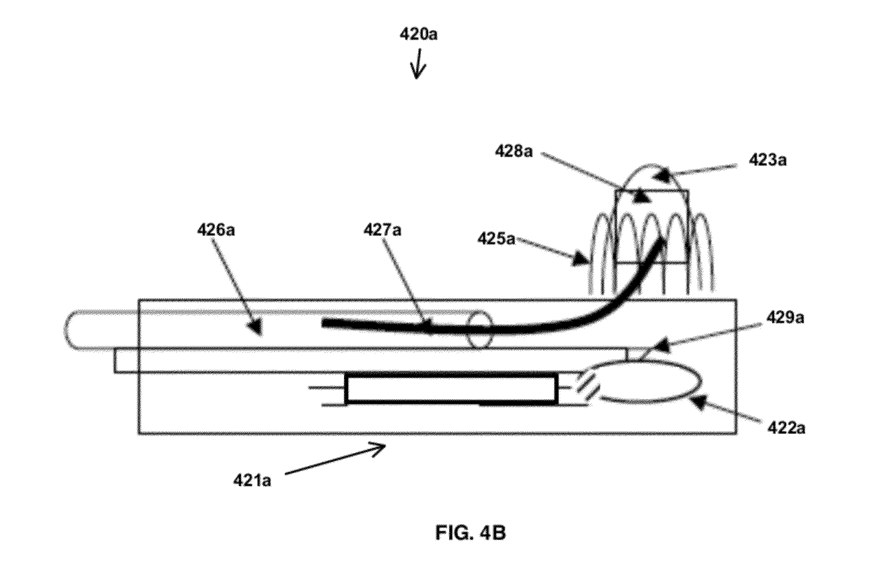

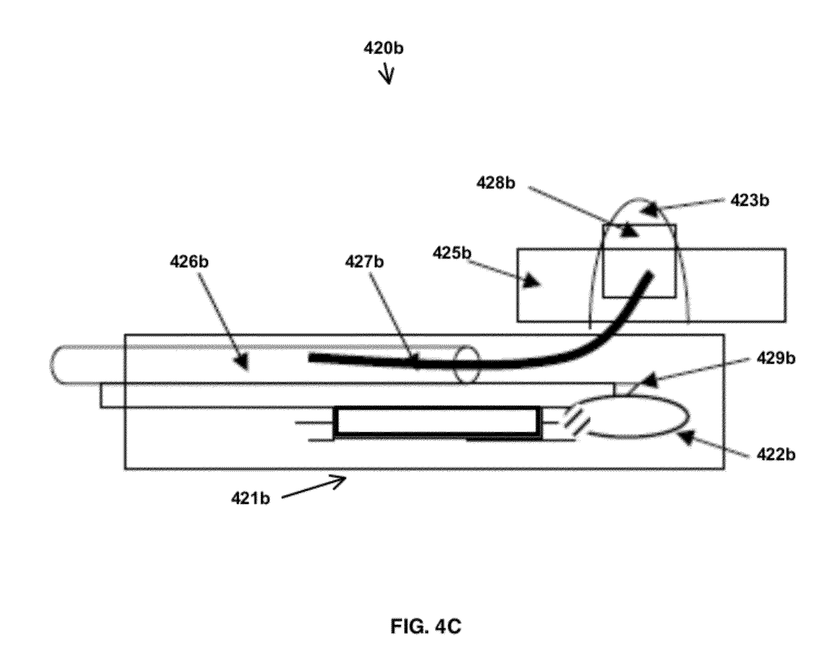

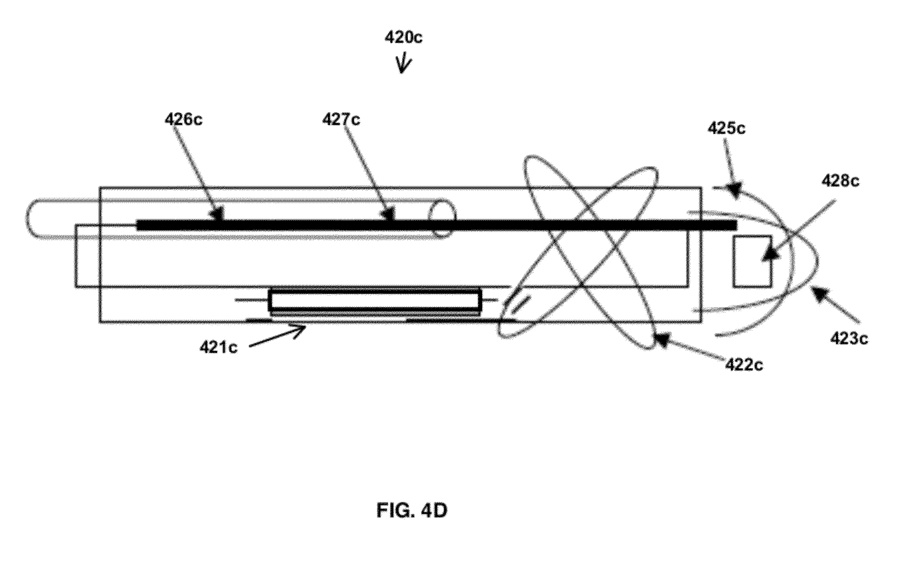

FIG. 4A depicts the general architecture of the primary elements of the RoboScanner device. The device comprises one or more sensors of Limited-FOV modalities 401 and appropriate lines 402 for transferring the raw signal(s) of the Limited-FOV modalities to the data acquisition units along 403. The device comprises an assembly of actuators 404 used for the mechanical scanning with the RoboScanner device. A channel 405 for performing localized biopsy, e.g. fine-needle aspiration, guided by the Limited-FOV and the Wide-FOV modalities and the unit 406 for the collection of multiple biopsy samples in containers that are in correlation to the specific position the sample is collected are included. Another channel 407 for locally delivering a contrast agent that can tag the particular pathologic locus for enhancing the Limited- and/or the Wide-FOV modalities or for delivering a therapeutic agent and a unit 408 for storing and delivering the agent also are included. The device further comprises position actuators 409 and fiducial markers 410 for the registration of the delivery device to the Wide-FOV modality. The device may be configured to have sideways 411 and/or forward 412 Limited-FOV imaging, biopsy and/or agent delivery access to the tissue.

FIGS. 4B-4D illustrate examples of probe designs utilized for multi-modal guided tissue sampling. Designs encompass a LIF+MR side-looking probe 420a, a OCT-MR side-looking probe 420b and a CoM+MR forward-looking probe 420c. The probes incorporate the same components including an access cannula, needle or catheter 421a,b,c, an RF coil 422a,b,c for localized Mr and/or fiducially marking, for example, with a tuning capacity to the frequency of operation and a co-axial cable for connection to the MR scanner RF interface. The sensitive area of the RF coil is represented at 423a,b,c which is the area from where the majority of the MR signal, e.g. 90%, originates and which can be calculated by the Biot-Savart law or experimentally determined. In 420a the LIF probe is shown at 424a and in 420b the OCT probe is shown at 429b. Each probe has a sensitive area of detection of fluorescence 425a,b,c. For each probe 420a,b,c, a multitude of fluorescence acquisitions can be performed and if needed averaged for covering a wider area of tissue, as this may be determined by the Limited-FOV modality with the larger FOV.

The probes also include a channel of access 426a,b,c for sampling physical tissue with its associated applicator 427a,b,c, for example, a bendable needle for performing fine needle aspirations. For a side-looking probe, the needle is bendable and its bend shape also may be controlled with a suitable mechanical or piezoelectric or other type of element. The design of the applicator is such that the FNA is performed at a volume of tissue 428a,b,c that is inside the area detected by the sensors, ensuring that there is a spatial coincidence of the area of tissue that gives riose to the signal and the area of tissue sampled.

In a multi-modal guided tissue sampling operation the sampling system is advanced to position Sj at step 432 and multimodal data lj is collected at 434 and, if needed, the raw data Ij is processed at 436. Processing may be automated at 438a or by manual operation at 438b and selection criteria are inputted at step 440. The system is queried at 438c whether to proceed to the site to sample the tissue. If No at 438d, the system moves to the next position Sj+1 at 418. If Yes at 438e, the sampling mechanism is actuated at 442 and the physical sample is collected at 444 and stored and indexed at 446, whereupon the system proceeds to the next position Sj+1 at 448.

An example of a simple code for actuating tissue sampling at 444 uses two criteria in an AND type selection. At 450, the signal from modality “1” is above a threshold and at 452 the signal from modality “2” is within a lower and upper limit. A query is made at 454. When both conditions 450 and 452 are true at 456 then the tissue sampling mechanism is actuated at 442. If one or both of conditions 450 and 452 are not met at 458, the system proceeds to the next position 448. The control code may also incorporate constraints of the minimal step between samples.

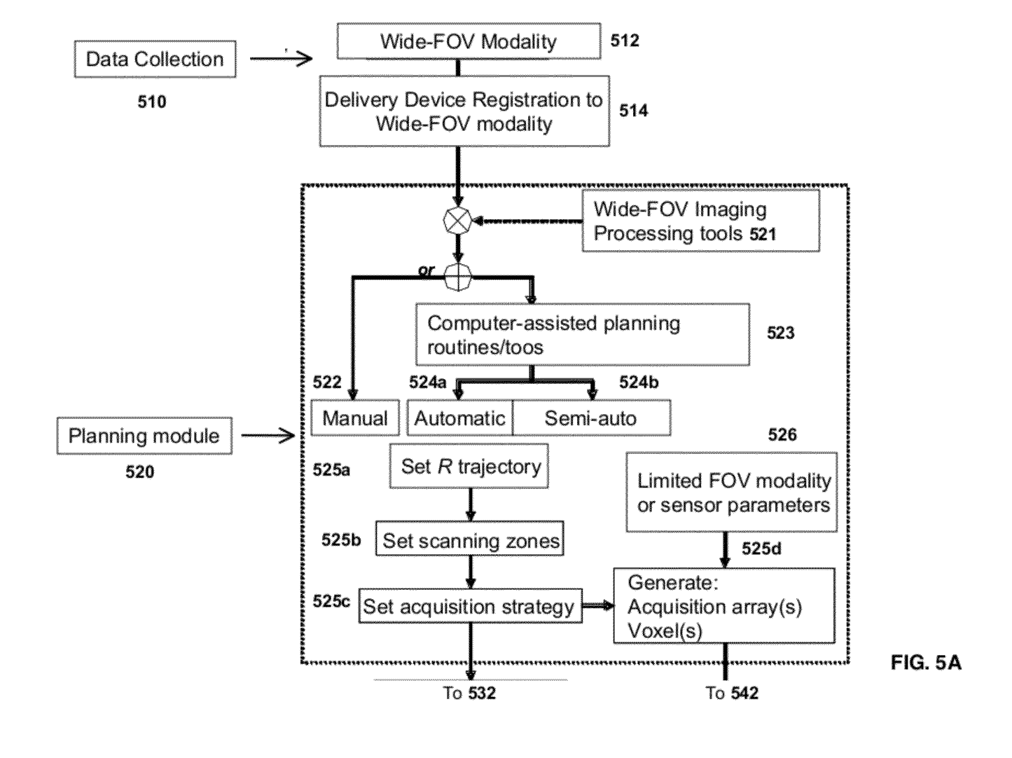

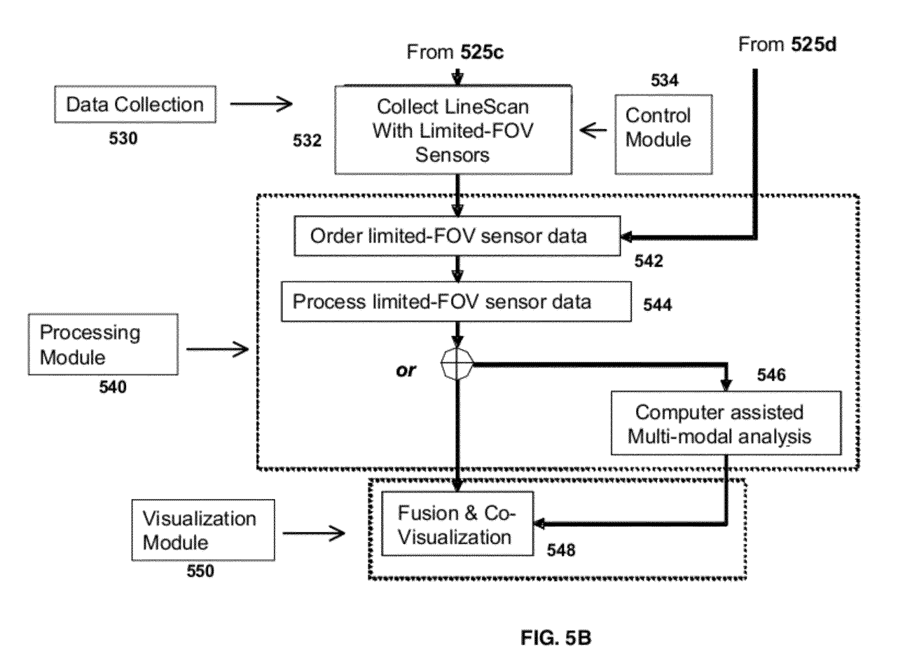

FIGS. 5A-5B show a flowchart of the operation of the RoboScanner system delineated the tasks and the module related to the particular task. The data collection module 510 enables collection of Wide-Foy modality data at 512 and registration of the delivery device to the Wide-FOV modality images at 514. This step can be performed at any point of the procedure but before the tasks in the planning module 520.

The planning module 520 enables the selective processing 521 of the Wide-FOV images to be appropriate for the planning task. The planning module also enables the collection of the Limited-FOV data. This task maybe performed entirely manually at 522 or via computer-assisted planning routines and/or tools at 523 that may be semi-automated 524a or automated 524b. This task entails the processes of setting the trajectory R of scanning 525a, setting the zones for scanning 525b, setting the acquisition strategy 525c, and calculating for the generation of the Acquisition Array (AA) 525d from the Limited-FOV modality or sensor parameters 526. The planning module allows the operator to manually or semi-automatically, or automatically define a trajectory for the insertion of the delivery device. This trajectory, can be configured to pass through the portion of the tissue identified from the Wide-FOV modality for scanning with the Limited-FOV sensor(s), for example, through a lesion, and may be arranged to avoid harming vital structures, for example, blood vessels or nerves, or to minimize the length of the traversed healthy tissue.

The data collection module 530 enables the collection 532 of the LineScan data with the Limited-FOV data based on the acquisition strategy 525c with overall control from the control module 534. The processing module 540 enables multi-modal processing that includes ordering and co-registration of the Limited- and Wide-FOV data 542, processing of the Limited-FOV data for extraction of information 544 and, selectively, additionally analysing for multi-modal interpretation 546, for example, but not limited to, data mining and machine learning processes. The visualization module 650 enables multi-modal visualization for diagnosis 548.

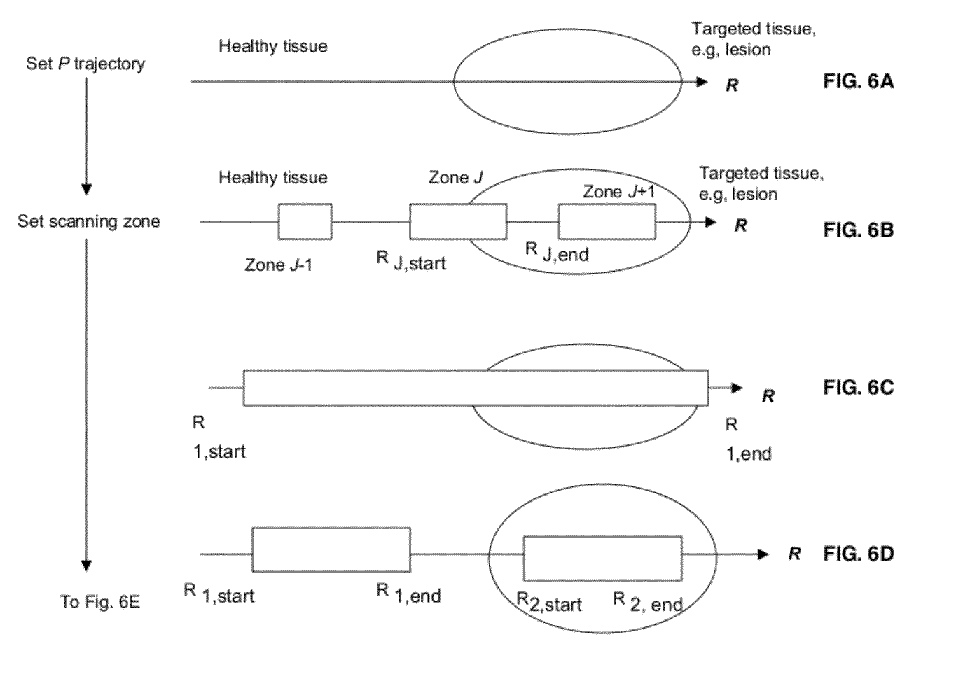

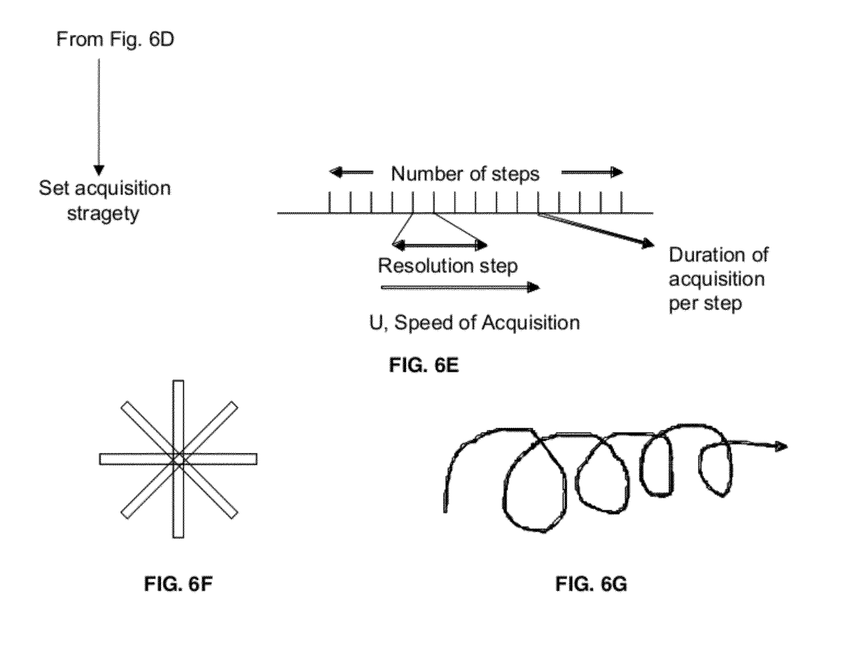

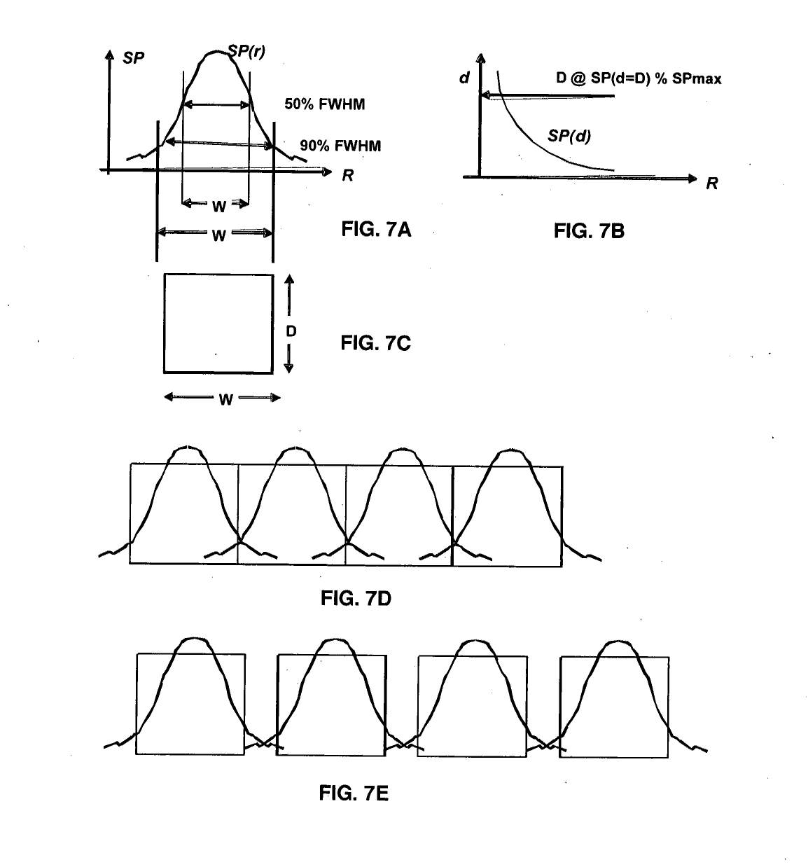

FIG. 6A schematically depicts setting the P trajectory where, using the Wide-FOV images, the insertion vector R is defined based on the selected spectroscopic or imaging method selected and the targeted tissue. FIGS. 6B-6D depict the selection of the scanning zones, which may be done manually or semi-automated or automated by the software and may include at least one zone on the tissue. FIG. 6B illustrates the assignment of three scanning zones along vector R. Zone 1 is placed on healthy tissue, Zone 2 is placed on the boundary of the healthy and targeted tissue and Zone 3 is placed on targeted tissue. FIG. 6C illustrates the assignment of one scanning zone along the vector R that covers the three tissue zones. FIG. 6D illustrates the assignment of two scanning zones along the vector R. Zone 1 is placed on healthy tissue and Zone 2 is placed on targeted tissue. FIG. 7E schematically depicts the general format of setting data acquisition along the R-vector. This entails identifying representative parameters that can be adjusted by the operator for the acquisition of Limited-FOV data. Different patterns of scanning can be implemented depending of the DoF of the delivery device, such as, but not limited to, of multiple planes around the R vector in FIG. 6F or a 3D spiral centred around the R-vector in FIG. 6G.

The system provides graphical and visualization tools that facilitates setting scanning zones and setting the acquision strategy. In a preplanned scanning strategy, the operator may select or plan a single continuous scanning or multiple areas to be scanned from the Wide-FOV data or images. The pre-planned scanning strategy can be selected automatically with image analysis software or images or can be selected manually by visual inspection and graphical tools from the Wide-FOV data or images,

FIGS. 7A-7E illustrate the size of the AA cell of “volume modality”, such as but not limited to LIF and non-volume selective MRS, and the definition of an acquisition voxel. In FIG. 7A, the Limited-FOV sensor has a detection profile SP(r). The width (W) of the voxel, centered at the current position of the center of detecting sensor, can be defined by the operator or automatically by the software as the Width-at-a-specific % of max detection. Example shown, but not limited to, at 50%, known as full width at half maximum=FWHM, and 90%. Any other widths can be then defined. In FIG. 7B, the Limited-FOV detection also depends on the depth the signal originates described by a SP(d) where d is the distance from the sensor. Depending on the characteristic of the modality and depending on parameters such as the available signal-to-noise a depth (D) can be determined that corresponds to a fifth of the SPmax. Then D is the distance from the sensor that the SP(d=D)=% SPmax. In FIG. 7C, the Voxel has a width W×D. In FIG. 7D, the SP profiles along the scanning axis and the associated voxels are illustrated. In FIG. 7E a different AA array is depicted where the acquisition positions have been separated further to reduce cross-voxel contamination.

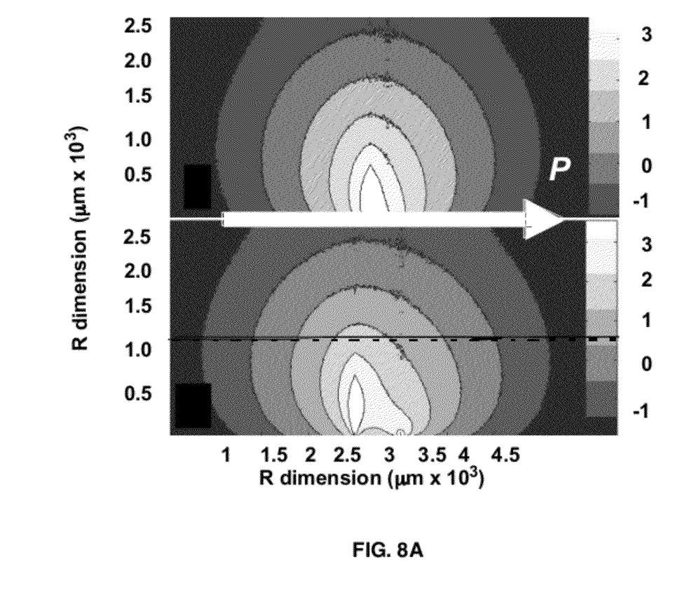

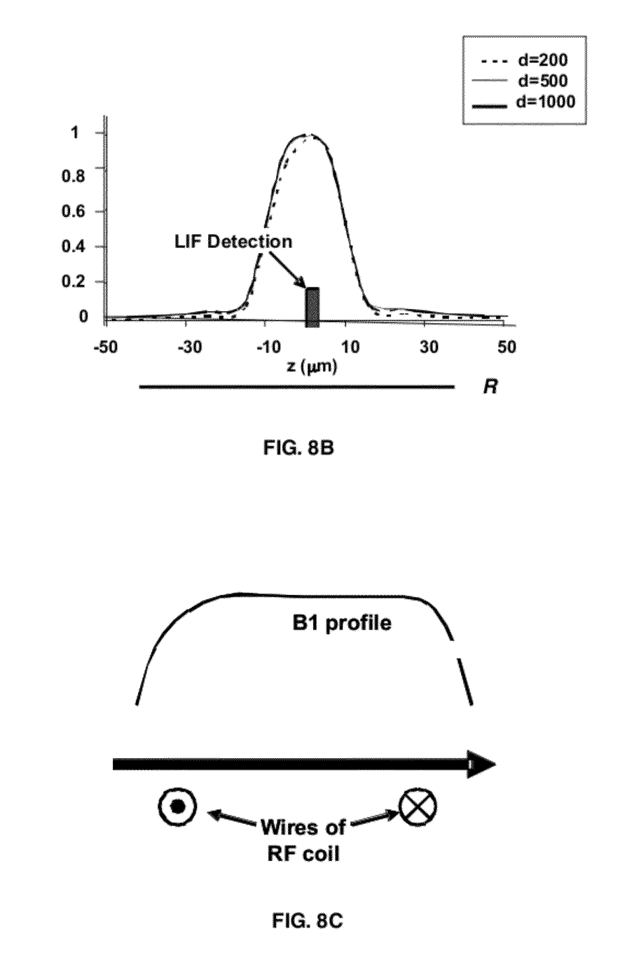

FIGS. 8A-8C show characteristic SP profiles for Volume Modalities. Spectroscopy, e.g., LIF, samples signals that can theoretically originate from a 3D volume, but in practice are limited by its SNR, the aperture of the detecting optical element and its distance to the light emitter. The aperture sets a 3D “width” along the axis of the detection cone, and the distance the “depth”. This 3D profile can be estimated experimentally or with numerical simulations via, for example, but not limited to, Monte Carlo simulations. In FIGS. 8A-8B, the detection area of a LIF sensor was performed using Monte Carlo simulations for source-detector distances of 200 um and 1000 um and the corresponding profiles are depicted on a Log Scale graph. In FIG. 8C, the B1-profile of a circular RF coil is depicted. Those SP(R) profiles are used by the processing module to generate the AA array and the voxel array.

Generally, FIGS. 8A-8C illustrates results of the detected fluorescence at a plane defined by the source/detector pair, and shows that its 2D detection profile along the R vector is rather independent from the source/detector distance. The graph shows the profile of the LIF detection along the axis of scanning identifying also the position of the LIF detection optical fiber. This profile illustrates the concept of the “width” of the corresponding AA_{J} voxel for the LIF Limited-FOV. The same concept applies to other optical spectroscopic modalities as well as for MR spectroscopy using a miniature RF coil as a sensor. Thus, for the OCT and variable geometry LIF FOV's to coincide, a straightforward approach is to place the collecting detectors at the same location on the probe axis. Since there is limited time for data acquisition, the collection of the same number of OCT and LIF acquisitions may not be appropriate, rather more OCT lines may be collected around the location of LIF collection. The acquisition protocols are analyzed.

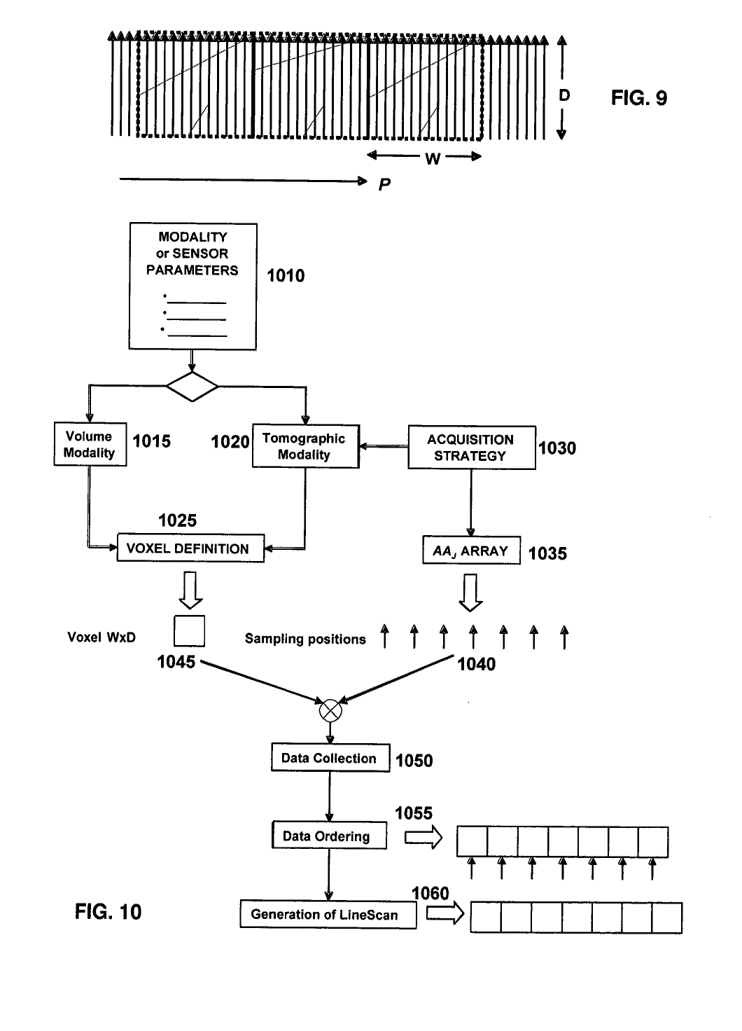

FIG. 9 depicts the size of the AA cell of the tomographic modality, such as but not limited to OCT. The AA size is determined by the number of lines and the depth by the inherent to the modality tissue penetration. Alternatively, the size of the AA cell of “volume modality” that is spatially localized due to the particular way it is collected, such as the Signal Voxel Spectroscopy MRS, is determined by the voxel size of the particular acquisition method.

FIG. 10 depicts the processes and interfaces from the planning to generating the LineScan. The modality or sensor parameters are identified at 1010 and used with either a volume modality 1015 or a tomographic modality 1020, both of which separately define the voxel 1025. The process for acquisition strategy 1030 interfaces with both the tomographic modality and the AAj array 1035 which comprises sampling positions data 1040. The voxel definition includes Voxel W×D data 1045 which is collected 1050 and stored 1055 together with the sampling positions data. This data is used in the LineScan generation process 1060.

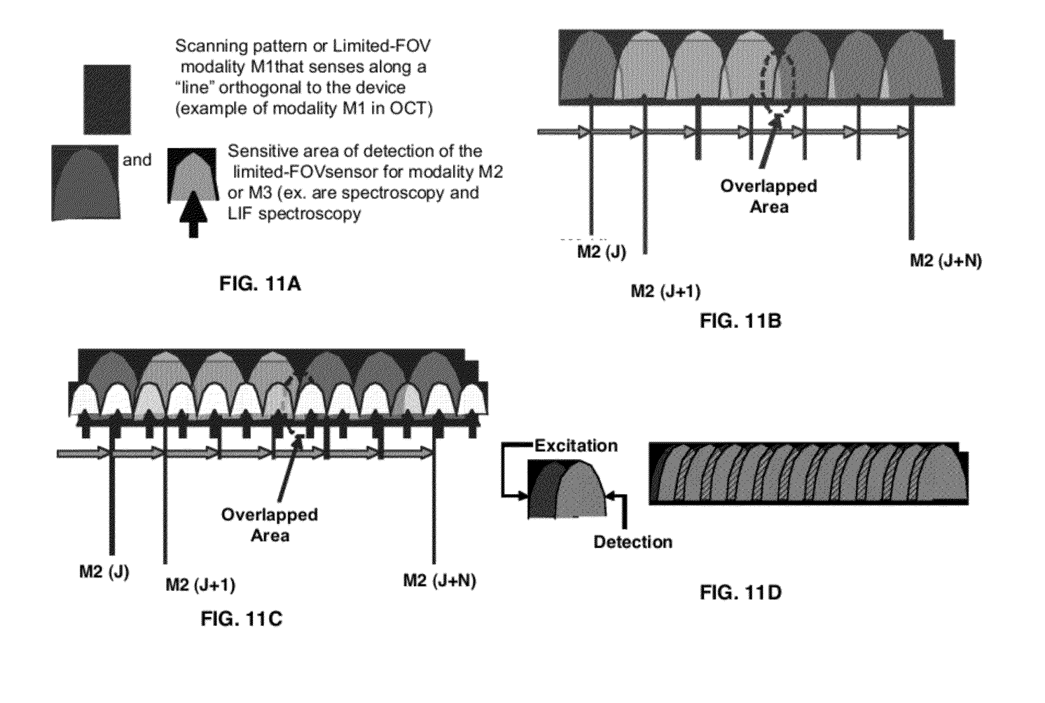

FIGS. 11A-11D illustrate different scanning patterns with the Limited-FOV modalities of the system, but not to scale. FIG. 11A depicts two examples of characteristic types of data collection with Limited-FOV sensors. The upper pattern is utilized with methods that collect data along a line orthogonal to the axis of the probe, such as Optical Coherence Tomography modality M1 and the lower pattern corresponds to methods that excite and detect signal from a volume of the tissue, as MR spectroscopy modality M2 and LIF modality M3.

FIG. 11B diagrams a method of scanning with the Limited-FOV sensors M1 and M2 using the device along a line that can be performed in multiple distinct steps with the sequence: (move from position A to position B and collect data of say modality M1)-(stop)-(collect data of modality M2)-(repeat until completing scanning). FIG. 11C diagrams a method of scanning and collecting data with the Limited-FOV sensor M1 and M2 using the device along a line that can be performed in a continuous fashion with the sequence (continuously move between the two positions A and B, while interleaving the collection of multiple modalities, e.g., modalities M1 and M2). The graphs shows M1 sensor for clarity. As depicted in FIG. 11D, due to continuous acquisition the excitation and detection profiles are not matching.

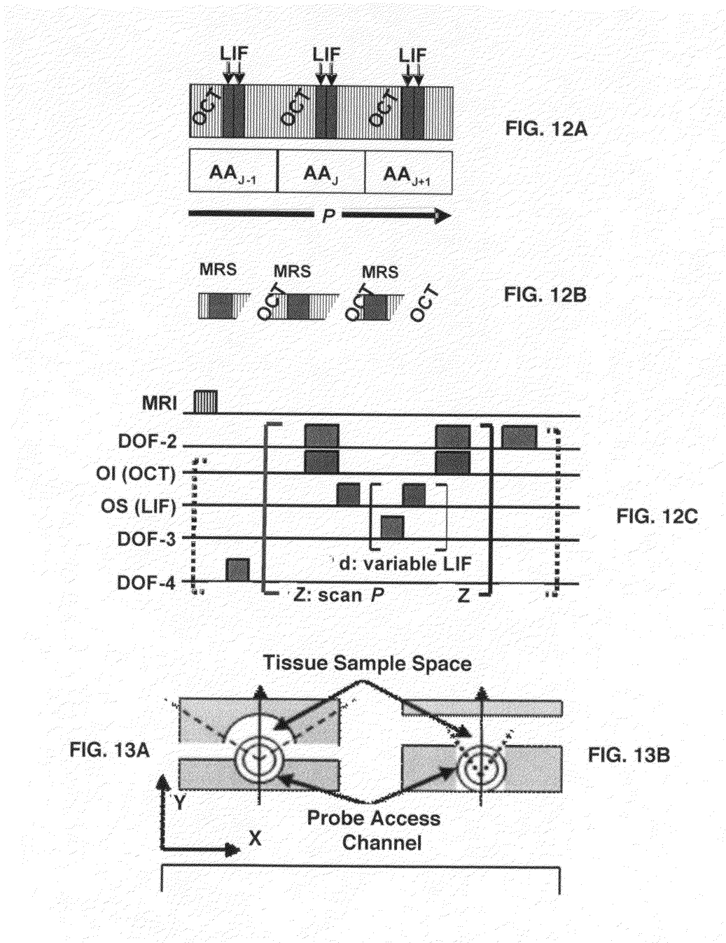

FIGS. 12A-12C show acquisition protocols. Specifically, FIG. 12A shows examples of optical probe scanning protocol for the streaming optical data collection that interleaves and spatially matches OCT and LIF detection to the center of the corresponding AA_{J} (acquisition array) cell and FIG. 12B shows the streaming-like collection of OCT interleaved with MRS. In FIG. 12C LIF is performed at two distinct instances per cycle, each corresponding to different distances of the emit/receive optical fibers.

FIGS. 13A-13B show two examples of tissue holders adapted to appropriately position the sample relative to the delivery device for imaging and/or biosensing of excised tissue for ex vivo studies. When the probe rotates around Z, with Type A (left) the sensors have the same distance from the sample, while Type B (right) maintains the sample flat.

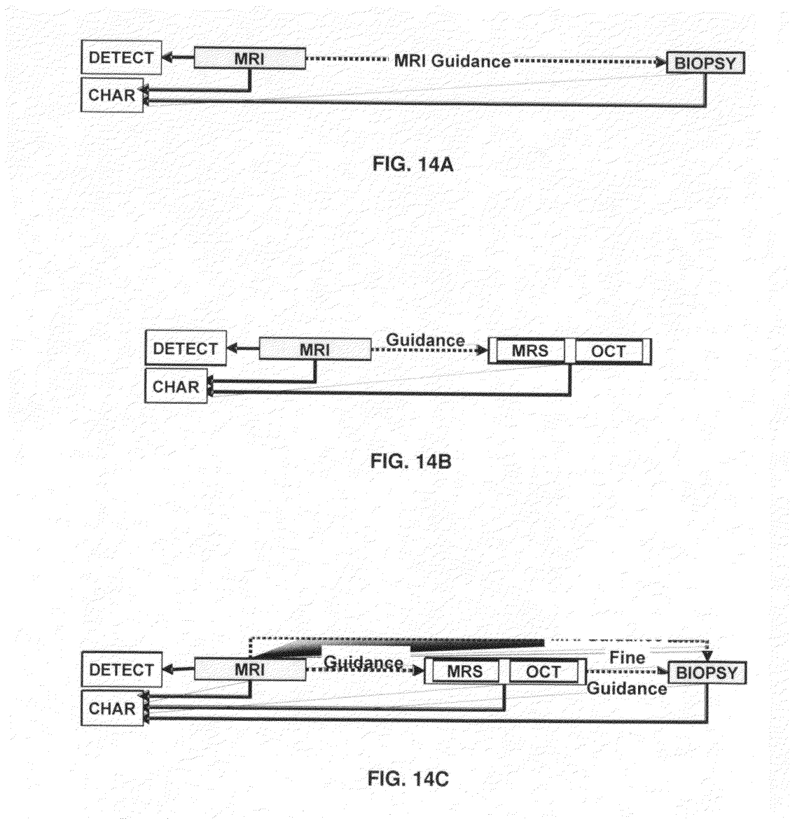

FIGS. 14A-14C depict a system designed as a complement to or as an alternative to standard tissue biopsies. Modern in situ biosensors may eventually evolve to alternatives to tissue biopsy, a possible clinical tool. If the system can provide high resolution tissue characterization then it may be used for fine guidance of tissue biopsies, potentially offering detailed definition of lesion boundaries and tissue inhomogeneities. lesion detection (DETECT) and characterization (CHAR), with MRI, OCT+MRS and tissue biopsy. The dashed arrows show which modality guides what. Solid lines show which modality (information) is used. FIG. 14A depicts the currently practiced MRI & MRI guided biopsy. FIG. 14B depicts a MRI & OCT+MRS guided biospsy, and FIG. 14C depicts the combination of OCT+MRS with biopsy; the former also used for fine guidance of biopsy sampling.

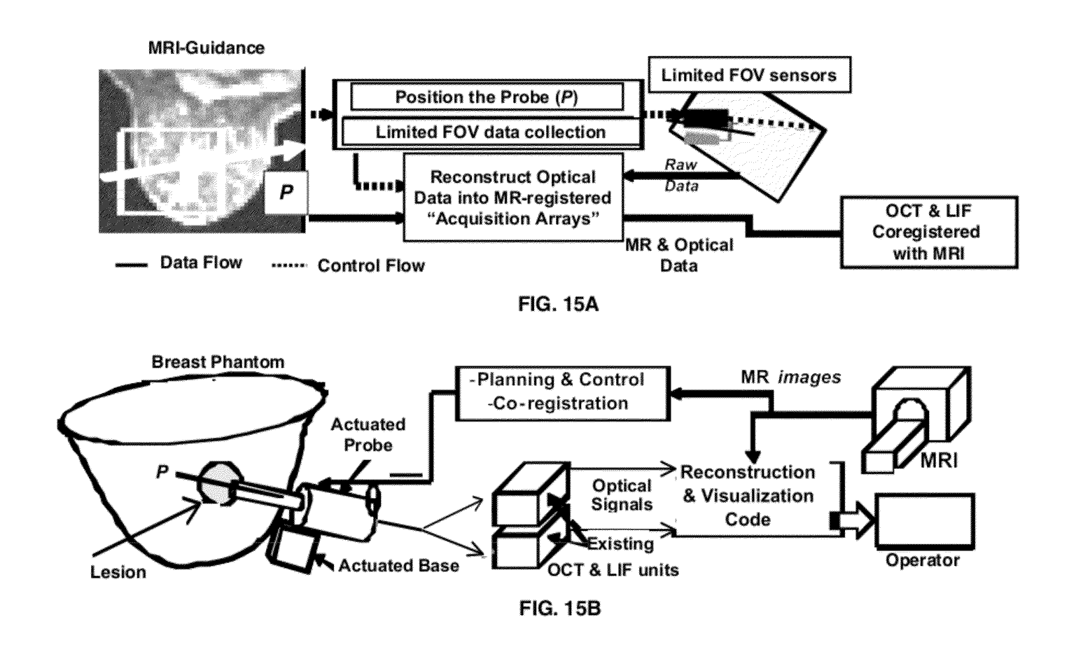

FIG. 15 discloses two implementations of the RoboScanner combines MRI as the Wide-FOV modality, and LIF and OCT as the Limited-FOV modalities. The system is shown for use in breast cancer scanning; but it can be used for other anatomical areas and pathologies, such as, but not limited to, prostate cancer. They demonstrate that a lesion can be detected in the breast.

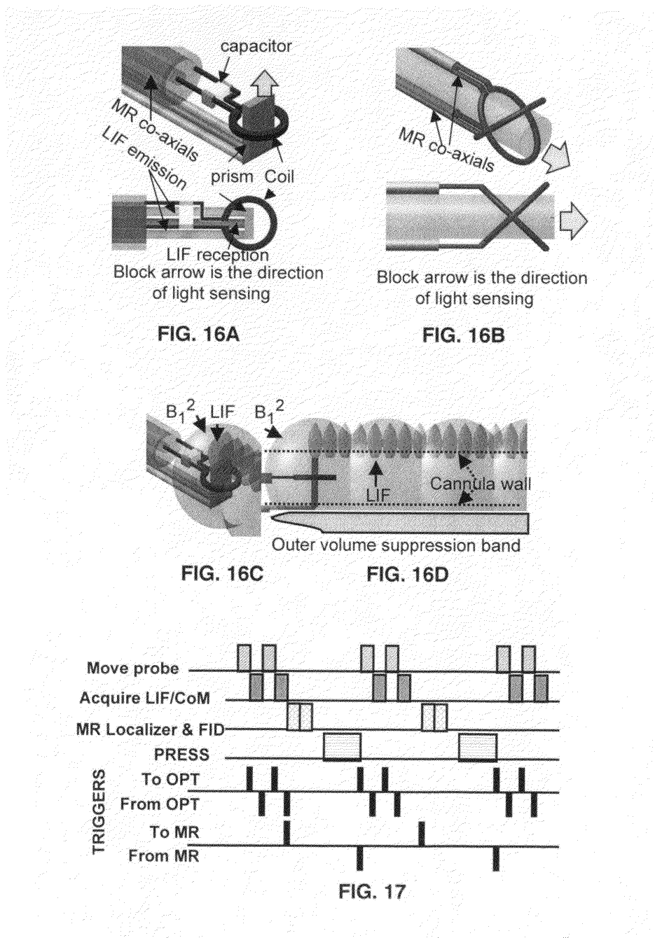

FIGS. 16A-16D are examples of dual sensor modalities. FIG. 161A depicts a side-looking laser/light-induced fluorescence (LIF) plus MR dual sensor and FIG. 16B depicts a forward-looking confocal microscopy plus MR dual sensor. In both cases the RF coils for MR are connected to the Rf interface of the MR scanner via ultra-thin co-axial cables and can be used as Receive only (Rx), Transmit only (Tx) or Transmit/receive (Tx/Rx). Alternatively, those coils can be implemented as inductively coupled to an external larger RF coil; this allows the probe to be implemented in a thinner form. In particular the coil of the CoM/MR case in FIG. 16B is implemented by means of two coils that are orthogonal to each other. Alternatively a single 8-figure coil can be implemented that operates in a linear fashion and allows a thinner factor. FIG. 16C demonstrates a 3D simulation of isosurface (99% of B1) and 95% of fluorescence superimposed to the LIF+MR probe depicted in FIG. 16A. FIG. 16D depicts an interleaved streaming-like collection of LIF interleaved to MRS. The semitransparent shaded area is an optional OVS

FIG. 17 depicts a timing diagram of LIF/CoM (OPT) and MRS data collection, including the associated triggering scheme.

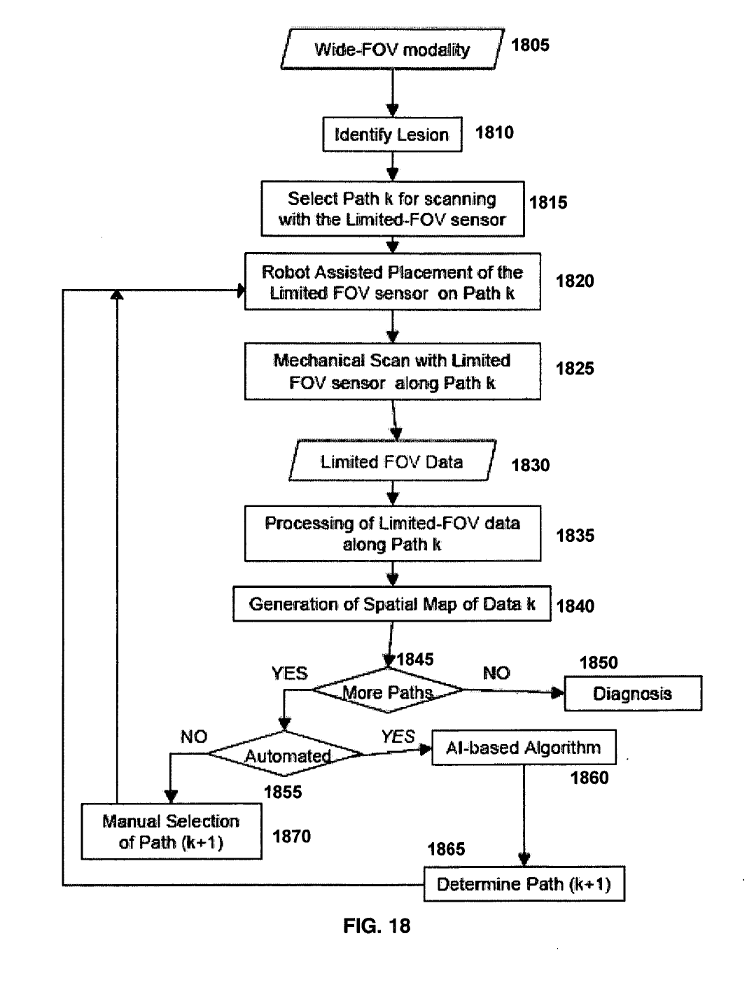

FIG. 18 is a flowchart illustrating a method for a multimodality and multilevel scan for tumors with one or more paths of scanning. First, the Wide-FOV modality is selected at 1805 and the lesion is identified at 1810. The scanning path (k) is selected for the Limited-FOV sensor at 1815. The Limited-FOV sensor is placed along the path via robotic assistance at 1820 and a mechanical scan is conducted of the sensor along the path at 1825. The Limited-FOV data is collected at 1830, processed along the path (k) at 1835 and the spatial map of the (k) data is generated at 1840. There is a query at 1845 if more paths are to be scanned. If No, a diagnosis is made at 1850. If Yes, and the system is automated at 1855, an algorithm is implemented at 1860 and the path (k+1) is determined at 1865. If Yes, and the system is not automated at 1855, a manual selection of path (k+1) is made by the operator at 1870. For either an automated or manual set-up, after path (k+1) is selected, the Limited-FOV sensor is placed along path (k+1) at 1820.

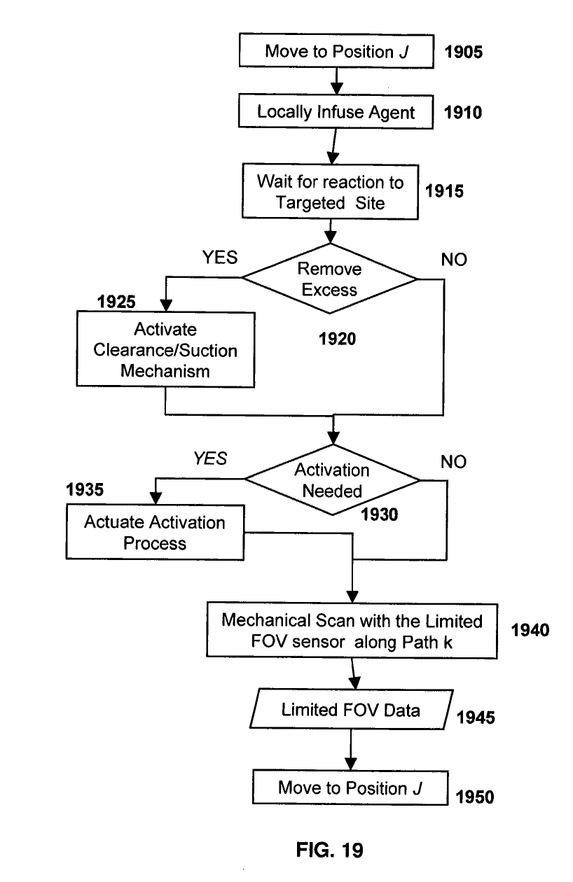

FIG. 19 is a flowchart for using the device for the local infusion of a contrast agent that may or may not require activation. The device may also allow the removal of excess contrast agent. The Limited-FOV is moved to position J at 1905. The contrast agent is infused locally at 1910 and allowed to react at the targeted site at 1915. The system is queried whether excess contrast agent must be removed at 1920. If No, the method proceeds to a query if activation is required at 1930. If Yes, a clearance/suction mechanism is activated at 1925 to remove excess contrast agent and the method proceeds to 1930. If no contrast agent activation is required, the system proceeds to mechanically scan the Limited-FOV along the path k starting at position J at 1940. If Yes, an activation process is actuated at 1935 and the method proceeds to the mechanical scan at 1940. The Limited-FOV data is collected at 1945 and the Limited-FOV sensor is moved to position J+1.

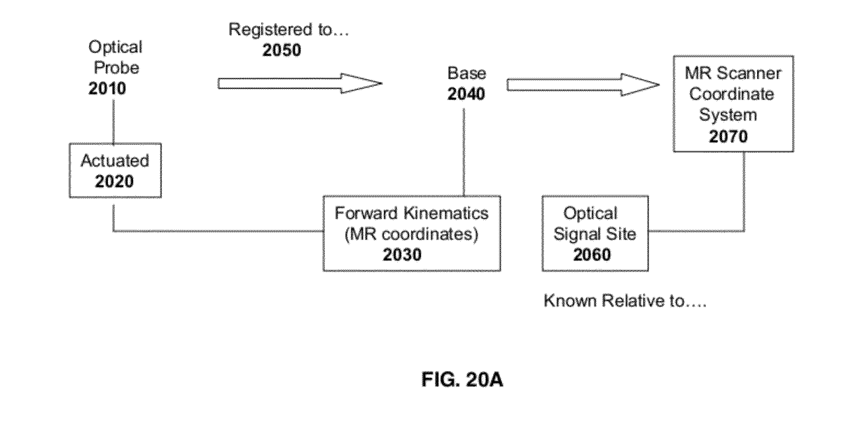

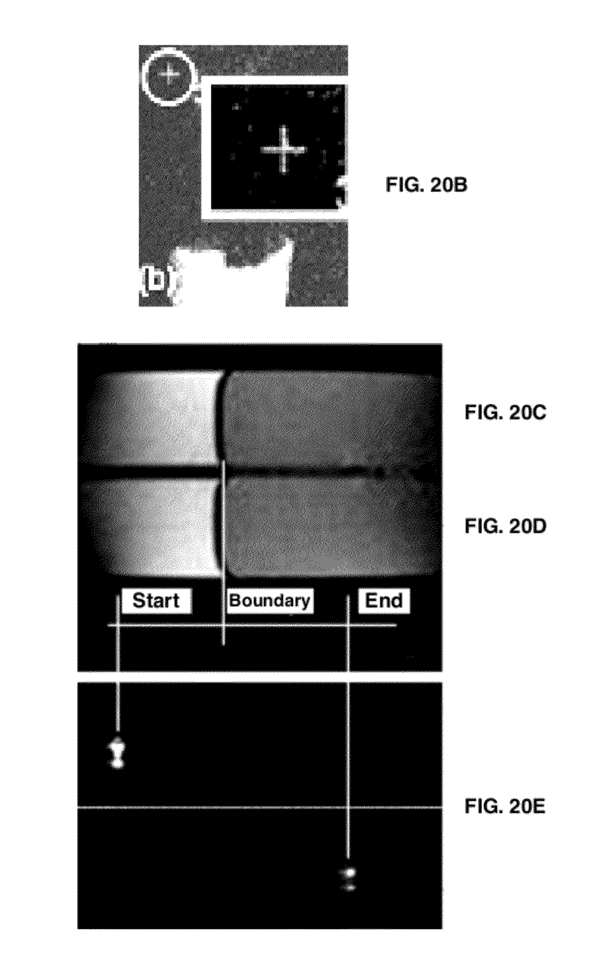

FIGS. 20A-20E illustrate the registration relationship and hierarchy of the MR-registered entities, from the probe that carries for Limited-FOV sensor) to the MR scanner. Based on these relationships, the collected Limited-FOV signals, e.g. the optical signals of the OCT or LIF sensors, are registered to MRI as discussed below. FIG. 20A depicts the process implemented for generating LineScans. The optical probe 2010 is actuated at 2020 and the forward kinematics of the MR coordinates 2030 are provided to the base 2040. The optical probe is also registered at 2050 to the base. Both the base and the optical signal site 2060 are linked to the MR scanner coordinate system 2070. FIG. 20B is an MR image of a cross-shaped fiducial marker made of an appropriate compartment filled with Gd-based contrast agent that can be used for the initial registration of the device. FIGS. 20C-20D show the registration results a GRE MRI image of a dual compartment phantom collected with the body RF coil of a scanner and two GRE images collected with the same FOV as in FIG. 20A but using the miniature RF coil, that is, the Limited-FOV sensor also is used as fiducial marker, at two different positions along the axis of the device. FIG. 20E shows the image collected at the most distal scanning position.

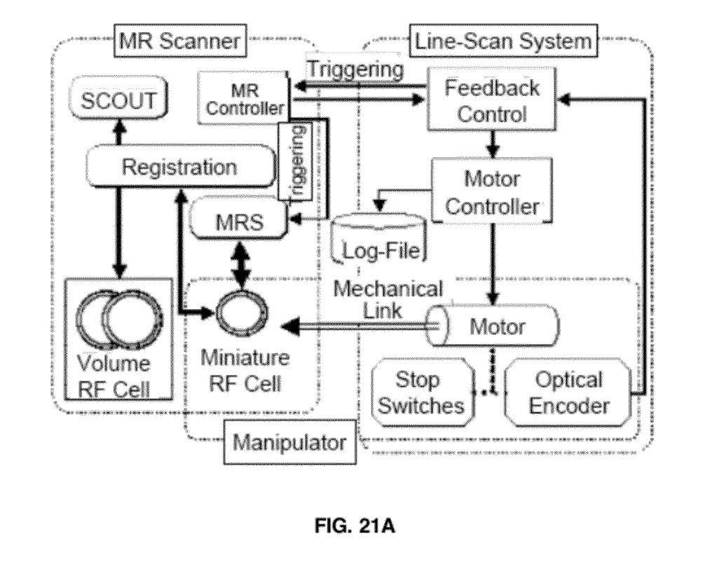

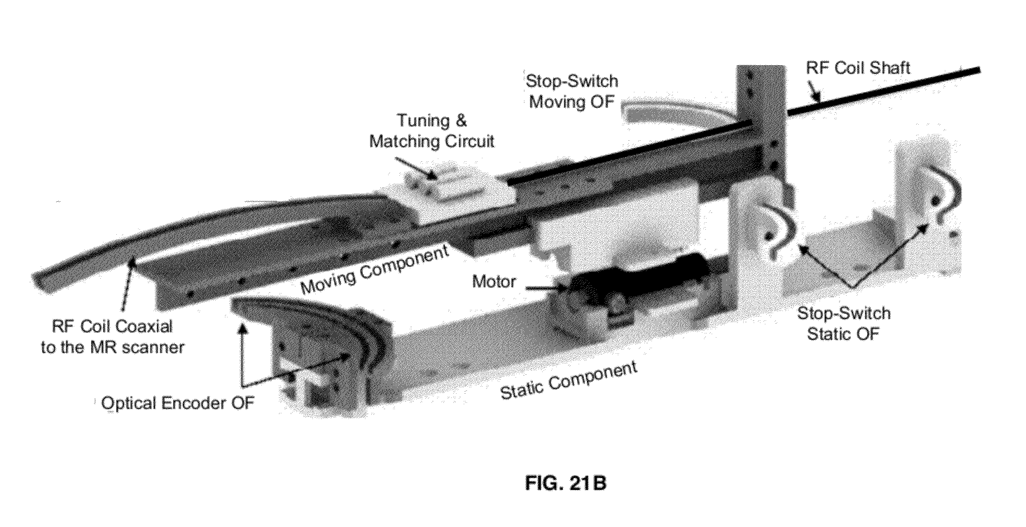

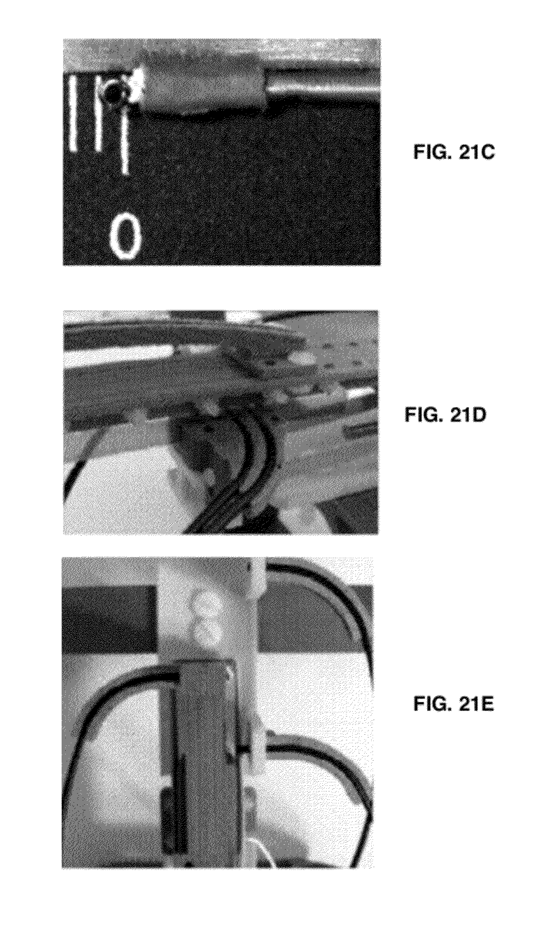

FIGS. 21A-21E illustrate the architecture of the system implemented for line-scanning. In FIG. 21A the architecture the line-scan system, the MR scanner and the manipulator. The line-scan system, i.e. the Limited-FOV modality, and the MR scanner, i.e. the guiding modality, are inter-connected via the mechanical link, i.e. the actuated manipulator. The system presented in FIGS. 21A-21E is a special implementation of the generalized system of FIG. 1. The manipulator shown in FIG. 21B carries and scans with the Limited-FOV sensor, i.e. the miniature RF coil, which is depicted in FIG. 21C. The motion of the sensor is registered to the MR scanner coordinate systems; as a result the positions that the sensor collects data with the Limited-FOV modality are registered to the guiding modality. A volume coil is used for imaging the area of interest thereby also simulating the operation MRI-guidance for the placement and planning of the line-scan.

At the line-scan system site all process and data management are performed by the control core shown in FIG. 21A that also communicates with the MR scanner controller via triggering pulses. Specifically, and to automate the line-scan data collection, a two-way triggering scheme is implemented. After the completion of each repositioning along the line-scan, as validated by the signals from the optical encoder, the control core generated a TTL pulse that is directed to the MR scanner controller. This pulse triggered the collection of an MR spectrum, i.e. at this particular locale. Upon conclusion of this particular data collection, the MR controller generated another TTL that triggered the advancement of the sensor to its next location. With this mechanism, the system could perform any type of desired unsupervised scanning protocol, for example, as presented in FIGS. 12A-12C. During a scan, the control core saves a series of data into a log-file, including the date and time that the MR scanner and the motor are triggered, and the coordinate of the transient position of the miniature RF coil on Z dimension. Several measures were adopted to ensure MR compatibility of the system. In brief, the system is made entirely of non-magnetic and non-conductive material, a piezoelectric commercial motor is used for actuation, all optical-fiber based linear encoder and stop switches were incorporated, and all electronic components were located outside the scanner's room and at over 6 meters away from the magnet.

The manipulator is first designed and its mechanical structure optimized using computer 3D solid modeling (Autodesk Inventor), and then physically prototyped entirely of non-magnetic and non-conductive acrylonitrile butadiene styrene (ABS). FIG. 21B shows a photograph of the RoboScanner manipulator. Detailed blueprints of the different parts were physically prototyped using a 3-D Fused Deposition Modeling printer (Prodigy Plus model, Stratasys, Eden Prairie, Minn.) out of ABS. The parts were then assembled to form the final manipulator. Actuation is performed with a Squiggle motor (New Scale Technologies) with 6 meters long shielded power wiring for placing its controller away from the MR scanner. As demonstrated in the Examples section, this motor proved sufficiently MR compatible.

The distal end of the manipulator carries a four turn miniature solenoid RF coil with a diameter of 1.1 mm and length of 1.2 mm, as shown in FIG. 21C, which is formed by manually winding 32 AWG shielded wire. The coil is connected via a 15-cm long 1.2 mm OD semi-rigid coaxial cable (Micro-Coax, Pottstown, Pa.) to a balanced matching and tuning circuit which made of non-magnetic variable capacitors (Johanson Manufacturing Co, NJ) for fine tuning and matching on the proton Larmor frequency of 201.5 MHz for operation at the employed 4.7 Tesla scanner.

The hardware set up is based on a PC that is connected via a serial port to the motor controller (MC-1000, New Scale Technologies Inc., Victor, N.Y.) and via a USB port to a data acquisition unit (DI-148U, DATAQ Instruments Inc., Akron, Ohio) for performing the dual-triggering scheme. The control core software is developed on Matlab (Mathworks, Inc., Natick, Mass.), using the ActiveX library for the motor controller (NstSquiggleCTRL ActiveX by New Scale Technologies) and the ActiveX control interface for communication with the data acquisition unit (UltimaSerial by DATAQ Instruments Inc.). The dual triggering scheme is implemented by running two co-axial cables from the data acquisition unit to the MR scanner controller.

To facilitate closed loop control, the manipulator is equipped with in-house developed MR-compatible optical sensors. A quadrature linear optical encoder assesses the translation of the probe, as FIG. 21D, and two stop-switches positioned at the rail of the moving sub-assembly, to hard-limit its movement within a range of 5 cm, primarily to prevent over-extending the motor, as shown in FIG. 21E. Both sensors were made fully MR-compatible by implementing them with light-only operation, and moving the electronics outside the magnet room. Light emitting diodes (LED) were used as light sources and photo-activated Schmitt triggers for generating pulses to sense changes in the light beams (LED were model IF E97 and the Schmitt trigger model Photologic Detector IF D95T both from Industrial Fiber Optics, Inc., Tempe, Ariz.). The circuit boards, located in the operator's room, were connected to the optical sensor sub-assemblies attached onto the manipulator using 7 meters long fiber optic cables (ESKA SH 3001 Mitsubishi Rayon Co., Japan).

The quadrature linear optical encoder in FIG. 21D operates based on standard principles of quadrature detection. In brief, light emitted from the LED is transferred via the optical fiber cables to the encoder sub-assembly that is fixed on the manipulator base and is static. The light passes through an encoder strip and via a return optical fiber reaches a photo-activated Schmitt trigger. The signal from the Schmitt trigger is directly fed to the quadrature decoder of the MC-1000 motor controller, stored into its register, and used in the closed loop. The encoder strip is attached onto the translating sub-assembly of the manipulator and thus modulates the light beam. It is composed of alternating 0.25 mm wide transparent and non-transparent bands (printed on a transparent plastic strip with a laser printer). The two lagged square wave signals, generated by the two Schmitt triggers, were then used to determine the extent and direction of the motion. The operation of the stop switches is based on the exact same principle of operation. Specifically, the light carrying optical fiber cable is attached onto the moving sub-assembly while two returning fibers are fixed at the extreme positions of the rail. When the light carrying fiber aligns with either one of the fixed returning cables, active low signal is generated by the TTL inverters; this blocks the motor.

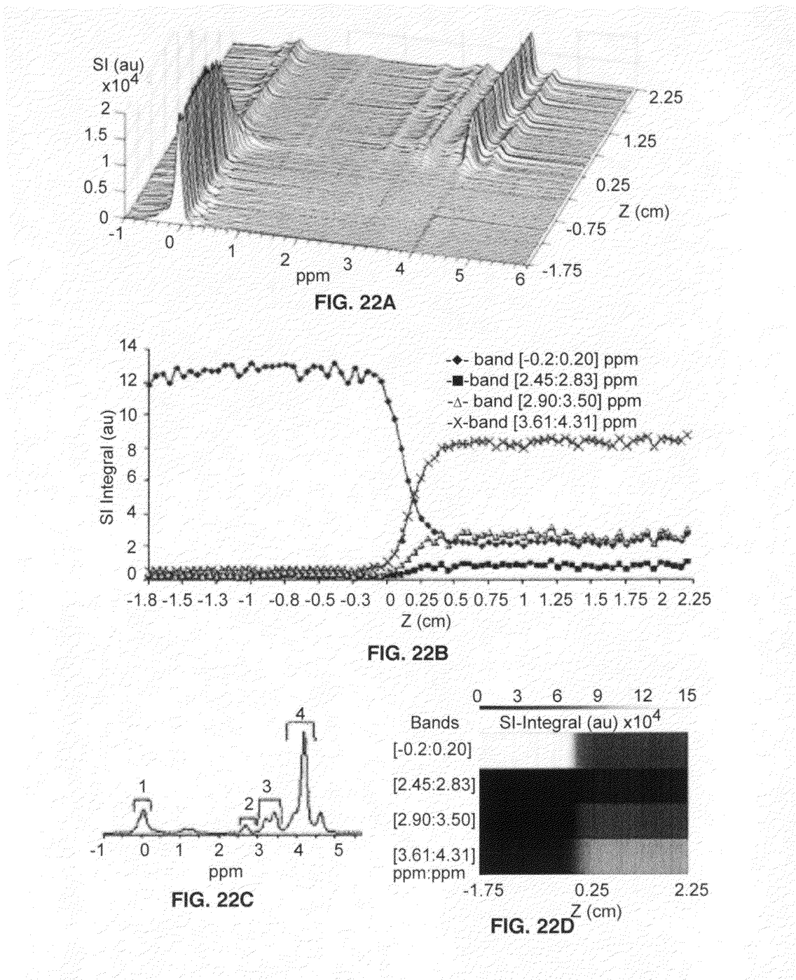

FIGS. 22A-22D show the results, further described in Example 2, from scans using a miniature RF coil as the Limited-FOV sensor. The scans are of a dual compartment phantom with gelatin and oil. FIG. 22A is a stacked single-pulse spectra full bandwidth in 3D. FIGS. 22B-22C are graphs of integrated intensity vs. Z for water, oil peak 1 and oil triplet peaks. FIG. 22D is pseudo-color map of the integrated intensities of bands at certain spectral densities.

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### Example 1

### Co-Registration of MRI and MRS

The essence of the proposed approach is the use of the actuated manipulator to mechanically couple and co-register the guiding, i.e., MRI, and the Limited-FOV, i.e., MRS, modalities. The adopted approach is based on the facts that the MR scanner has its inherent coordinate system defined by the resident magnetic field gradient coils and any MR signal generating entity can be imaged relative to this coordinate system.

Specifically, this is performed by registering an initial position of the probe to the MR coordinate system and then by calculating its transient position based on the known motion steps from the optical encoder. FIG. 20A shows the approach used for registering the RF coil and the miniature RF coil is used to collect an image (FIG. 20B) at the initial position. From this image the exact coordinate of the sensor can be extracted and if desired compared with the image collected with the large volume RF coil (FIG. 120C vs. FIG. 20D). Any other position can also be registered as shown in FIG. 20E where the probe can be advanced to its most distal scanning position.

This approach has been successfully used before in MR-guided robot control (155), with the difference that instead of the optical encoder signals used herein, in that work it is used the outcome of the forward kinematics. Practically, this is implemented with a straightforward process: (a) the exact initial position (Z₀) of the probe relative to the coordinate system of the MR scanner is measured, (b) during line-scanning, the transient position (Z_{N}) of the probe is calculated based on the optical encoder signal counts (±N; including direction relative to the MR coordinate system) and the pre-set step of motion (DZ); i.e. Z_{N}=Z₀+N·DZ. It should be noted that due to the specific experimental set up, the probe is translated only along the Z axis of the magnet; in the general case the above approach can be used for translation, i.e. scanning, along an arbitrary vector.

The initial registration of the manipulator is performed using the miniature RF probe as a fiducial marker (FIG. 21D). The use of fiducial markers, with or without dedicated RF coils to enhance the marker's MR signal, is a well developed and used practice in interventional MRI. FIG. 20B illustrates the example of a dedicated cross-like fiducial marker used before for registering a surgical robot relative to the MR scanner.

Herein, instead of a separate dedicated marker, the miniature RF coil. i.e. the Limited-FOV modality sensor, is employed to collect a GRE sagittal image to image along the Z-axis with a Wide-FOV. The projection of the image is then calculated and the center of the RF coil profile is selected as the Z₀. Instead of a complete image, the projection along any axis of translation can be collected with the appropriate MR pulse sequence. The full image is preferred for better visualization of the probe in those preliminary studies. Alternative to this process would be the collection of an image or a projection after any translation to calculate the transient sensor position.

### Example 2

### Line Scan System

### Data Collection

All MR studies are performed on a ^{UNITY}/NOVA (Varian, Palo Alto, Calif.) spectrometer imager system. The sample holder, that carries the phantom, is secured onto a custom-made base, the probe is inserted into the scanning channel, and the assembly is then inserted into the volume RF coil and secured onto the scanner's cradle. The cradle is positioned so the centre of the volume coil corresponded to the isocenter of the scanner, and then both the volume and the miniature RF coils are fine tuned and matched with the cradle in place. This or a similar set-up can be used in phantom, excised tissue and animal studies.

Studies are performed using two-compartment rectangular phantoms (39.6×44.6×89 mm³), one filled with a gelatin matrix and the other with commercially available vegetable oil. The sample holder, that carries the phantom, is secured onto the custom-made base, the probe is inserted into the scanning channel, and the assembly is then inserted into the volume RF coil and secured onto the scanner's cradle. The cradle is positioned so the center of the volume coil corresponded to the isocenter of the scanner, and then both the volume and the miniature RF coils were fine tuned and matched with the cradle in place.

MR compatibility studies evaluated the effect of the presence and operation of the manipulator on ¹H spectra, collected after a single excitation pulse, i.e. the free induction decay (FID), and on gradient recalled echo (GRE) images. To better appreciate the effect of motor operation, dynamic acquisition studies were performed. At these studies GRE images and spectra were continuously collected. After a period of motor idling, to collect baseline data, the manipulator is actuated. The specific acquisition parameters for the GRE and the FID were the same with those used in the line-scans, reported in the following paragraph. Those studies were performed with both the volume and the miniature RF coils.

Line-scan MR data collection studies were performed with the following protocol. Scout and multislice imaging with a gradient recalled echo (GRE) sequence (TR/TE/a; matrix, FOV), shimming on a 5 cm wide slab along the axis of the scanning and localized scout spectroscopy with point resolved spectroscopy (PRESS) (bandwidth=4006.4 Hz number of points=8000 and voxel size=64 mm³). The device is registered by collecting an appropriate slice orientation, for example, a sagittal image using the miniature coil with a GRE (same parameters as above). The initial position of the translating element is calculated as discussed in this invention. Subsequently a scanning protocol is prescribed and the Line-scan is performed with the automated procedure described in section A by repeated translation-spectra collection steps. Usually eighty to ninety steps of 0.5 mm were performed. After each translation has been completed, the control software triggered the spectrometer that collected the free induction decay (FID) after a single excitation pulse (with a flip angle=45°; bandwidth=5000 Hz and number of points=2048).

### Processing MRI/MRS Collected Data

All data processing is performed off-line with in-house developed software on Matlab. For each set of experimental data, the initial position of the miniature RF coil, for example, as shown in the FIGS. 21A-21E architecture, is determined from the MR images collected with it. Subsequently, the acquisition strategy file that is stored during data collection is loaded, the positions where spectra were collected were extracted and then stored in an array (Z₁, where I=1 to N_{MAX}). This array is then used for registering and sorting spectra collected with the miniature coil. Those spectra are arranged on a 2D matrix S_{MIN-I,J} (I=1 to N_{MAX} and J=1 to N_{A}+2) for further processing; two additional rows were used, one to store the Z_{I} and the other the frequency in ppm).

The spectra are Fourier transformed and the zero ppm frequency is assigned at the water peak from the gelatin compartment. Resonances are first identified on the PRESS spectra collected with the volume coil and then on representative S_{MIN} spectra collected at center of the phantom compartments prescribing the bands of each resonance. For each identified peak, the software extracted the following: center resonance in ppm and difference in Hz from the water peak, integrated intensity, SNR and reported them as value±standard deviation. Additional outputs included stacked plots of the spectra and graphs of parameter vs. position on the Z axis. The latter are further processed to identify the position Z_{SP} as mid-way of the transition zone. Images collected with the volume coil were also loaded and the position (Z_{IM}) of the boundary between the two compartments is extracted and compared to this calculated from the spectra, i.e. Z_{SP}.

### Line Scan Results

The device demonstrated sufficient MR compatibility with insignificant effect on the SNR of the images, and the SNR and line width of the spectra. Specifically, with the motor idling the gradient recalled echo images manifested a SNR for the gelatin compartment of 7.08±0.10 and vegetable oil of 5.72±0.07. When the motor is operating at its set speed, the SNR of the gelatin is 6.79±0.11 and of the oil compartment is 5.62±0.06. The SNR of spectra is measured to be 2260±36 for the water and 1076±28, for the most explicit oil resonance, for the motor idling. When the motor is actuated the SNR of those resonances is measured 579±26 for the gelatin and 830±31 for the most explicit oil resonance.

FIG. 22A illustrates a line-scan set presented in the form of stacked plots of single-pulse spectra collected every 0.5 mm along the Z axis of the scanner. The transition from the gelatin filled compartment to this with vegetable oil is apparent at Z=1.5 mm. This is manifested with the reduction of the water signal and the emergence of the signals originating from the oil compartment. It is notable that the spectra manifest a transition zone between the two compartments, i.e. the water and oil signals change gradually. This is due to the fact that the miniature coil excites and receives signal from an area that is wider than 0.5 mm. The line-scan and the transition between the two compartments can also be appreciated in the topographic representation of this set in FIG. 22C, where the presented bandwidth of the spectra has been reduced to 1400 Hz (from −1 ppm to 6 ppm).

To further appreciate the spatial localization using the miniature coil with a single pulse sequence, FIGS. 22B-22C shows the integrated intensity of the five identified resonances vs. the position of the coil along the Z axis. These graphs clearly illustrate the presence of the boundary between the two compartments, as well as the existence of the above mentioned transition zone between them. FIG. 22D shows a pseudo-color map of the integrated intensities of the bands of certain spectral widths. This is an example of an output of the RoboScanner system for visualizing the results. The center of the transition zones is the same for the five signals. It is also noted that the assignment of the Z coordinates is based on the initial registration of the device and then using the recorded motion values, calculated from the linear encoder recordings, and saved into the log-file. The Z axis in FIGS. 22A and 22C is also assigned in the same manner.

The following references are cited herein.
1. Yang et al. Ann Nucl Med, Vol. 20, pp. 1-11, January 2006.
2. Gibson et al. Phys Med Biol, Vol. 50, pp. R1-43, Feb. 21, 2005.
3. S. Achilefu, Technol Cancer Res Treat, Vol. 3, pp. 393-409, August 2004.
4. R. G. Blasberg, Mol Cancer Ther, Vol. 2, pp. 335-43, March 2003.
5. Boppart et al. Neurosurgery, Vol. 43, pp. 834-41, October 1998.
6. R. M. Hoffman, “Apmis, Vol. 112, pp. 441-9, July-August 2004.
7. S. Sathornsumetee & J. N. Rich, Anticancer Drugs, Vol. 17, pp. 1003-16, October 2006.
8. Saleem et al. Eur J Cancer, Vol. 42, pp. 1720-7, August 2006.
9. Czernin et al. Annu Rev Med, Vol. 57, pp. 99-118, 2006.
10. K. Shah & R. Weissleder, NeuroRx, Vol. 2, pp. 215-25, April 2005.
11. Persigehl et al. Abdom Imaging, Vol. 30, pp. 342-54, May-June 2005.
12. Jager et al. Cancer Imaging, Vol. 5 Spec No A, pp. S27-32, 2005.
13. F. D. Rollo, Radiol Manage, Vol. 25, pp. 28-32; quiz 33-5, May-June 2003.
14. Gurfinkel et al. Dis Markers, Vol. 19, pp. 107-21, 2003.
15. Margolis et al. Radiology, Vol. 245, pp. 333-56, November 2007.
16. Zuiani et al. J Exp Clin Cancer Res, Vol. 21, pp. 89-95, September 2002.
17. Myaing et al.Opt Lett, Vol. 31, pp. 1076-8, Apr. 15, 2006.
18. Whiteman et al. Clin Cancer Res, Vol. 12, pp. 813-8, Feb. 1, 2006.
19. Sharwani et al. J Photochem Photobiol B, Vol. 83, pp. 27-33, Apr. 3, 2006.
20. Lane et al. J Biomed Opt, Vol. 11, p. 024006, March-April 2006.
21. Zhu et al. Neoplasia, Vol. 5, pp. 379-88, September-October 2003.
22. U. Mahmood & R. Weissleder, Mol Cancer Ther, Vol. 2, pp. 489-96, May 2003.
23. Shakhov et al. J Surg Oncol, Vol. 77, pp. 253-8, August 2001.
24. R. M. Hoffman, Biotechniques, Vol. 30, pp. 1016-22, 1024-6, May 2001.
25. Gurjar et al. Nat Med, Vol. 7, pp. 1245-8, November 2001.
26. C. Balas, IEEE Trans Biomed Eng, Vol. 48, pp. 96-104, January 2001.
27. White et al. Surgery, Vol. 128, pp. 1088-1100; discussion 1100-1, December 2000.
28. Pitris et al. J Gastroenterol, Vol. 35, pp. 87-92, 2000.
29. Fujimoto et al. Neoplasia, Vol. 2, pp. 9-25, January-April 2000.
30. Svanberg et al. Acta Radiol, Vol. 39, pp. 2-9, January 1998.
31. Anidjar et al. J Urol, Vol. 156, pp. 1590-6, November 1996.
32. Mankoff et al. Nucl Med Biol, Vol. 34, pp. 879-85, October 2007.
33. D. A. Mankoff, J Nucl Med, Vol. 48, pp. 18N, 21N, June 2007.
34. D. A. Mankoff, Breast Cancer Res, Vol. 10 Suppl 1, p. S3, 2008.
35. D. A. Mankoff, Q J Nucl Med Mol Imaging, Vol. 53, pp. 181-92, April 2009.
36. Mankoff et al. J Nucl Med, Vol. 49 Suppl 2, pp. 149S-63S, June 2008.
37. Celda et al. Adv Exp Med Biol, Vol. 587, pp. 285-302, 2006.
38. Ntziachristos et al. Proc Natl Acad Sci USA, Vol. 97, pp. 2767-72, Mar. 14, 2000.
39. Ntziachristos et al. Neoplasia, Vol. 4, pp. 347-54, July-August 2002.
40. Hsiang et al. Technol Cancer Res Treat, Vol. 4, pp. 549-58, October 2005.
41. Cleary et al. IEEE Trans Inf Technol Biomed, Vol. 6, pp. 249-61, December 2002.
42. F. A. Jolesz, Neurosurg Clin N Am, Vol. 16, pp. 201-13, January 2005.
43. Bugaj et al. J Biomed Opt, Vol. 6, pp. 122-33, April 2001.
44. Chen et al. Bioconjug Chem, Vol. 16, pp. 1264-74, September-October 2005.
45. Achilefu et al. Invest Radiol, Vol. 35, pp. 479-85, August 2000.
46. S. E. Harms, Semin Ultrasound CT MR, Vol. 19, pp. 104-20, 1998.
47. S. E. Harms & D. P. Flamig,” Clin Imaging, Vol. 25, pp. 227-46, 2001.
48. M. Sabel & H. Aichinger, Phys Med Biol, Vol. 41, pp. 315-68, March 1996.
49. Smith et al. Radiol Manage, Vol. 26, pp. 16-24; quiz 25-7, July-August 2004.
50. Behrenbruch et al. Med Image Anal, Vol. 7, pp. 311-40, September 2003.
51. S. Sinha and U. Sinha, NMR Biomed, Vol. 22, pp. 3-16, January 2009.
52. Singh et al. Future Oncol, Vol. 4, pp. 501-13, August 2008.
53. M. Tozaki and E. Fukuma, AJR Am J Roentgenol, Vol. 193, pp. 840-9, September 2009.
54. Sardanelli et al. AJR Am J Roentgenol, Vol. 192, pp. 1608-17, June 2009.
55. Morse et al. NMR Biomed, Vol. 22, pp. 114-27, January 2009.
56. Haddadin et al. NMR Biomed, Vol. 22, pp. 65-76, January 2009.
57. Baek et al. Int J Cancer, Vol. 123, pp. 1219-21, Sep. 1, 2008.
58. Mon et al. Cancer Res, Vol. 67, pp. 11284-90, Dec. 1, 2007.
59. Bolan et al. Magn Reson Med, Vol. 50, pp. 1134-43, December 2003.
60. Chang et al. IEEE Trans Med Imaging, Vol. 16, pp. 68-77, February 1997.
61. Brooksby et al. J Biomed Opt, Vol. 10, p. 051504, September-October 2005.
62. Hielscher et al. Dis Markers, Vol. 18, pp. 313-37, 2002.
63. Ntziachristos et al. Breast Cancer Res, Vol. 3, pp. 41-6, 2001.
64. Ntziachristos et al. IEEE Trans Med Imaging, Vol. 20, pp. 470-8, June 2001.
65. Choe et al. Med Phys, Vol. 32, pp. 1128-39, April 2005.
66. Boverman et al. Phys Med Biol, Vol. 50, pp. 3941-56, Sep. 7, 2005.
67. D. M. Agnese, Surg Technol Int, Vol. 14, pp. 51-6, 2005.
68. Elmore et al. Jama, Vol. 293, pp. 1245-56, Mar. 9, 2005.
69. Fantini et al. Technol Cancer Res Treat, Vol. 4, pp. 471-82, October 2005.
70. McNichols et al. in Proceedings of SPIE Biomedical Diagnostic, Guidance, and Surgical-Assist Systems III. Vol. Volume 4254, W. S. G. Tuan Vo-Dinh, David A. Benaron, Ed., 2001, pp. 23-30.
71. Sapozhnikova et al. in Proceedings of SPIE-Coherence Domain Optical Methods and Optical Coherence Tomography in Biomedicine VII, Vol. Volume 4956, J. A. I. Valery V. Tuchin, James G. Fujimoto, Ed., 2003, pp. 81-88.
72. Tumlinson et al. Proceedings of SPIE—Volume 4956 Coherence Domain Optical
Methods and Optical Coherence Tomography in Biomedicine VII, J. A. I. Valery V. Tuchin, James G. Fujimoto, Ed., 2003, pp. 129-138.
73. Tumlinson et al. Appl Opt, Vol. 43, pp. 113-21, Jan. 1, 2004.
74. Mountford et al. NMR Biomed, Vol. 22, pp. 54-64, January 2009.
75. D. P. Soares and M. Law, Clin Radiol, Vol. 64, pp. 12-21, January 2009.
76. Thomas et al. NMR Biomed, Vol. 22, pp. 77-91, January 2009.
77. Umbehr et al. Eur Urol, Vol. 55, pp. 575-90, March 2009.
78. Weinreb et al. Radiology, Vol. 251, pp. 122-33, April 2009.
79. K. Brindle, Nat Rev Cancer, Vol. 8, pp. 94-107, February 2008.
80. Callot et al. Eur J Radiol, Vol. 67, pp. 268-74, August 2008.
81. Kim et al. Korean J Radiol, Vol. 10, pp. 535-51, November-December 2009.
82. J. Kurhanewicz & D. B. Vigneron, Magn Reson Imaging Clin N Am, Vol. 16, pp. 697-710, ix-x, November 2008.
83. M. A. McLean & J. J. Cross, Br J Neurosurg, Vol. 23, pp. 5-13, February 2009.
84. Sardanelli et al. Radiol Med, Vol. 113, pp. 56-64, February 2008.
85. Thomas et al. MAGMA, Vol. 21, pp. 443-58, November 2008.
86. A. A. Tzika, “Int J Oncol, Vol. 32, pp. 517-26, March 2008.
87. Zakian et al. Cancer Biomark, Vol. 4, pp. 263-76, 2008.
88. Costanzo et al. Eur Radiol, Vol. 17, pp. 1651-62, July 2007.
89. Sibtain et al. Clin Radiol, Vol. 62, pp. 109-19, February 2007.
90. R. J. Young & E. A. Knopp, “Brain MRI: tumor evaluation,” J Magn Reson Imaging, Vol. 24, pp. 709-24, October 2006.
91. Bolan et al. Magn Reson Med, Vol. 48, pp. 215-22, August 2002.
92. Bolan et al. Breast Cancer Res, Vol. 7, pp. 149-52, 2005.
93. Corum et al. Magn Reson Med, Vol. 61, pp. 1232-7, May 2009.
94. Meisamy et al. Radiology, Vol. 233, pp. 424-31, November 2004.
95. Meisamy et al. Radiology, Vol. 236, pp. 465-75, August 2005.
96. Al-Safar et al. Cancer Res, Vol. 66, pp. 427-34, Jan. 1, 2006.
97. Beloueche-Babari et al. Cancer Res, Vol. 65, pp. 3356-63, Apr. 15, 2005.
98. Glunde et al. Neoplasia, Vol. 8, pp. 758-71, September 2006.
99. Lenkinski et al. Magn Reson Med, Vol. 61, pp. 1286-92, June 2009.
100. Ross et al. Mol Cancer Ther, Vol. 7, pp. 2556-65, August 2008.
101. M. Tozaki, “Breast Cancer, Vol. 15, pp. 218-23, 2008.
102. E. O. Aboagye & Z. M. Bhujwalla, Cancer Res, Vol. 59, pp. 80-4, Jan. 1, 1999.
103. Glunde et al. Magn Reson Med, Vol. 48, pp. 819-25, November 2002.
104. Katz et al. MAGMA, Vol. 6, pp. 44-52, August 1998.
105. Jagannathan et al. Br J Cancer, Vol. 84, pp. 1016-22, Apr. 20, 2001.
106. Gribbestad et al. Anticancer Res, Vol. 19, pp. 1737-46, May-June 1999.
107. Sitter et al. NMR Biomed, Vol. 19, pp. 30-40, February 2006.
108. Mackinnon et al. Radiology, Vol. 204, pp. 661-6, September 1997.
109. Cheng et al. J Magn Reson, Vol. 135, pp. 194-202, November 1998.
110. L. Bartella & W. Huang, Radiographics, Vol. 27 Suppl 1, pp. S241-52, October 2007.
111. Gee et al. Radiology, Vol. 248, pp. 925-35, September 2008.
112. Josephson et al. Bioconjug Chem, Vol. 13, pp. 554-60, May-June 2002.
113. Kircher et al. Cancer Res, Vol. 63, pp. 8122-5, Dec. 1, 2003.
114. U. Mahmood & L. Josephson, Proc IEEE Inst Electr Electron Eng, Vol. 93, pp. 800-808, April 2005.
115. Montet et al. Radiology, Vol. 242, pp. 751-8, March 2007.
116. Sheth et al. J Biomed Opt, Vol. 14, p. 064014, November-December 2009.
117. Upadhyay et al. Radiology, Vol. 245, pp. 523-31, November 2007.
118. Tearney et al. Science, Vol. 276, pp. 2037-9, Jun. 27, 1997.
119. Luo et al. Technol Cancer Res Treat, Vol. 4, pp. 539-48, October 2005.
120. Boppart et al. Breast Cancer Res Treat, Vol. 84, pp. 85-97, March 2004.
121. Brezinski et al. Heart, Vol. 77, pp. 397-403, May 1997.
122. Fujimoto et al. Ann N Y Acad Sci, Vol. 838, pp. 95-107, Feb. 9, 1998.
123. Bouma et al. Acad Radiol, Vol. 9, pp. 942-53, August 2002.
124. Sokolov et al. Technol Cancer Res Treat, Vol. 2, pp. 491-504, December 2003.
125. Breslin et al. Ann Surg Oncol, Vol. 11, pp. 65-70, January 2004.
126. Palmer et al. Photochem Photobiol, Vol. 78, pp. 462-9, November 2003.
127. Zhu et al. Biomed Opt, Vol. 10, p. 024032, March-April 2005.
128. Zeng et al. Opt Lett, Vol. 29, pp. 587-9, Mar. 15, 2004.
129. van Veen et al. Phys Med Biol, Vol. 50, pp. 2573-81, Jun. 7, 2005.
130. van Veen et al. J Biomed Opt, Vol. 9, pp. 1129-36, November-December 2004.
131. Mokbel et al. Eur J Surg Oncol, Vol. 31, pp. 3-8, February 2005.
132. Sardanelli et al. Radiology, Vol. 235, pp. 791-7, June 2005.
133. Nattkemper et al. Med Image Anal, Vol. 9, pp. 344-51, August 2005.
134. Oshida et al. Eur Radiol, Vol. 15, pp. 1353-60, July 2005.
135. Behrenbruch et al. Br J Radiol, Vol. 77 Spec No 2, pp. S201-8, 2004.
136. Jacobs et al. J Magn Reson Imaging, Vol. 21, pp. 23-8, January 2005.
137. Brix et al. Magn Reson Med, Vol. 52, pp. 420-9, August 2004.
138. Van Goethem et al. Eur Radiol, Vol. 14, pp. 1363-70, August 2004.
139. Jacobs et al. Radiology, Vol. 229, pp. 225-32, October 2003.
140. Szabo et al. Acta Radiol, Vol. 44, pp. 379-86, July 2003.
141. Degani et al. Thromb Haemost, Vol. 89, pp. 25-33, January 2003.
142. Kvistad et al. Radiology, Vol. 216, pp. 545-53, August 2000.
143. Helbich et al. Magn Reson Med, Vol. 44, pp. 915-24, December 2000.
144. Kvistad et al. Acta Radiol, Vol. 40, pp. 45-51, January 1999.
145. Kuhl et al. Radiology, Vol. 202, pp. 87-95, January 1997.
146. Daniel et al. Acad Radiol, Vol. 4, pp. 508-12, July 1997.
147. D.C. Zhu & M. H. Buonocore, Magn Reson Med, Vol. 50, pp. 966-75, November 2003.
148. Mahfouz et al. Eur Radiol, Vol. 11, pp. 965-9, 2001.
149. Gui et al. J Magn Reson Imaging, Vol. 24, pp. 1151-8, November 2006.
150. Zhu et al. J Biomed Opt, Vol. 8, pp. 237-47, April 2003.
151. Skala et al. Lasers Surg Med, Vol. 34, pp. 25-38, 2004.
152. Yu et al. Optics Express, Vol. 15, pp. 7335-7350, 2007.
153. Zhu et al. J Biomed Opt, Vol. 13, p. 034015, May-June 2008.
154. Palmer et al. Appl Opt, Vol. 45, pp. 1072-8, Feb. 10, 2006.
155. Christoforou et al. Magn Reson Imaging, Vol. 25, pp. 69-77, January 2007.

The present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered or modified and all such variations are considered within the scope and spirit of the present invention. Also, the terms in the claims have their plain, ordinary meaning unless otherwise explicitly and clearly defined by the patentee.

One of ordinary skills in the art, with the benefit of this disclosure, would recognize the extension of the approach to other fields of medical imaging and in particular in the field of multimodality imaging for assessing the pathophysiology of tissue in situ for diagnostic, therapeutic, and interventional, including surgical, procedures.

What is claimed is:

1. An automated system for multimodality imaging of a tissue in a subject, comprising:
a robotic delivery device configured for mechanically scanning the area of interest;
a computational core comprising a plurality of software modules electronically interlinked with the delivery device;
one or more interfaces between one or both of the computational core and the delivery device and an operator thereof;
at least one limited field of view sensor modality mechanically linked to or carried on the robotic delivery device and electronically linked to the computational core; and
at least one wide field of view imaging modality electronically linked to the computational core.

2. The automated system of claim 1, wherein the robotic delivery device comprises:
one or more probes configured to link one or more limited field of view sensor modalities to the area of interest;
means for dimensionally translating the one or more sensors along one or more axes of the delivery device; and
an acquisition unit for acquiring sensor data during translation thereof.

3. The automated system of claim 2, wherein the robotic delivery device further comprises a medium for coupling the one or more sensor modalities to the tissue.

4. The automated system of claim 2, wherein the robotic delivery device further comprises means for image-guided tissue sampling mechanically linked to or carried on the robotic delivery device and electronically linked to the computational core.

5. The automated system of claim 2, wherein the robotic delivery device further comprises one or more channels configured to:
accommodate means for delivery associated with a type of the device;
deliver locally one or more therapeutic agents, one or more diagnostic agents or a combination thereof;
locally deliver one or more contrast agents compatible with the sensor modality or the imaging modality; or
remove one or more fluids obstructing the link between the sensor modality and the tissue.

6. The automated system of claim 5, wherein the robotic delivery device type is a needle, a catheter or an add-on component to another interventional or surgical device.

7. The automated system of claim 5, wherein the contrast agent is activatable or targetable or a combination thereof.

8. The automated system of claim 5, wherein the contrast agent is activatable and the channel associated therewith is electronically linked to a means for activating the contrast agent disposed on the delivery device or external thereto.

9. The automated system of claim 1, wherein the limited field of view sensor modality comprises one or more of optical coherence tomography, light induced fluorescence, confocal microscopy, high-resolution ultrasound, or MR spectroscopy with miniature radiofrequency coils tuned to appropriate nuclei.

10. The automated system of claim 1 wherein the wide field of view imaging modality comprises magnetic resonance imaging (MRI), non-digital or digital x-ray, mammography, computer tomography (CT), and 2D or 3D ultrasound.

11. The automated system of claim 1, wherein the plurality of software modules comprise in electronic communication:
a planning module configured for planning collection of multimodal data;
a processing module configured for analyzing the collected data;
a tissue sampling module configured for sampling at one or more sites of interest on or in the tissue;
a visualization module configured for graphically outputting the analyzed data;
a fusion module configured for co-registering and co-visualizing of the one or more sensor modalities and the one or more imaging modalities;
and
a control module configured for linking the delivery device to the computational core.

12. The automated system of claim 11, wherein the planning module is configured to execute tasks to:
process the one or more imaging modalities;
determine scanning zones for the one or more sensor modalities based on the one or more processed imaging modalities;
determine a trajectory vector R for the scanning zones; and
implement a sensor modality data acquisition.

13. The automated system of claim 11, wherein the processing module is configured to execute tasks to:
order the data collected from the one or more sensing modalities;
process the ordered data; and
implement analysis of multimodal data.

14. The automated system of claim 11, wherein the tissue-sampling module is configured to execute tasks to:
determine one or more signal values received at one or more positions in one or more scanning zones during scanning of the sensor modality;
enable sampling or biopsy of the tissue at the one or more positions based on the signal values.

15. The automated system of claim 11, wherein the fusion module is configured to execute tasks to:
generate voxels or tissue volumes from data collected from the one or more sensor and imaging modalities;
produce spatial maps of the collected data; and
present the mapped spatial data to the visualization module.

16. The automated system of claim 11, wherein the control module is configured to execute tasks to:
control scanning;
actuate the collection of data with the wide field of view imaging modality and the limited field of view sensor modality according to the collection plan;
actuate tissue sampling and manage the tissue samples; and
deliver one or more of a contrast agent, therapeutic agent or diagnostic agent.

17. The automated system of claim 11, wherein the contrast agent is activatable, said control module further configured to execute the task to engage means for activation of the contrast agent.

18. A method for multimodal image scanning of a tissue, comprising the steps of:
selecting at least two modalities and co-registering the same;
selecting an area of interest on or in the tissue;
scanning the area of interest via the co-registered modalities; and
analyzing multimodal data collected during scanning.

19. The method of claim 18, further comprising obtaining a tissue sample at the area of interest for analysis.

20. The method of claim 18, further comprising delivering focally one or more of a therapeutic, diagnostic or contrast agent prior to or during scanning.

21. The method of claim 18, further comprising visually displaying the analyzed data as one or more spatial maps.

22. The method of claim 18, further comprising diagnosing a pathophysiological condition based on the analyzed multimodal data.

23. The method of claim 22, wherein the pathophysiological condition is a cancer.

24. The method of claim 18, wherein prior to the scanning step, the method comprises:
selecting at least one imaging modality as the two or more modalities;
determining one or more zones for scanning based on the at least one selected imaging modality;
determining a trajectory vector R for each of the at least one scanning zones;
selecting at least one sensor modality as the two or more modalities; and
implementing acquisition of sensor modality data.

25. The method of claim 18, wherein the step of analyzing the multimodal data comprises:
generating voxels or tissue volumes from the multimodal data; and
producing spatial maps of the collected data representing each scanned position comprising the one or more scanning zones.

26. The method of claim 18, wherein the at least two modalities comprise at least one wide field of view imaging modality and at least one limited field of view sensor modality.

27. The method of claim 26, wherein the limited field of view modality comprises one or more of optical coherence tomography, light induced fluorescence, confocal microscopy, high resolution ultrasound, or MR spectroscopy or imaging with miniature radiofrequency coils tuned to appropriate nuclei.

28. The method of claim 26, wherein the wide field of view imaging modality comprises magnetic resonance imaging (MRI), non-digital or digital x-ray, mammography, computer tomography (CT), and 2D or 3D ultrasound.

29. The method of claim 18, wherein the tissue is imaged in situ, in vivo or ex vivo.

30. The method of claim 18, wherein imaging the tissue comprises imaging a tumor or imaging a biochemical comprising the tissue.

31. A multimodal tissue scanning system, comprising:
a robotic delivery device configured to deliver a scanning signal from at least one sensor modality to one or more scanning zones on or in the tissue and to collect data during scanning, said one or more scanning zones determined from one or more selected imaging modalities; and, electronically linked to the delivery device:
a planning module configured to plan collection of multimodal data during scanning;
a processing module configured to analyze the collected data;
a visualization module configured to graphically output the analyzed data;
a fusion module configured to co-register all the modalities and co-visualize the analyzed scanning data; and
a control module configured to link the delivery device to the modules.

32. The multimodal tissue scanning system of claim 31, further comprising means for image-guided tissue sampling mechanically linked to or carried on the robotic delivery device and electronically linked to a tissue sampling module configured to enable sampling or biopsy of the tissue at the one or more scanning zones.

33. The multimodal tissue scanning system of claim 31, further comprising one or more of:
a medium for coupling the scanning signal to the tissue;
a channel to deliver locally to the tissue one or more therapeutic agents, one or more diagnostic agents or a combination thereof; or
a channel to locally deliver to the tissue one or more contrast agents compatible with the one or more modalities.

34. The multimodal tissue scanning system of claim 31, said system configured to dimensionally translate the one or more sensor modalities along one or more axes of the delivery device.

35. The multimodal tissue scanning system of claim 31, wherein the system is configured to image tissue in situ, in vivo or ex vivo.

36. The multimodal tissue scanning system of claim 31, wherein the one or more sensor, modalities comprise optical coherence tomography, light induced fluorescence, confocal microscopy, high resolution ultrasound, or MR spectroscopy with miniature radiofrequency coils tuned to appropriate nuclei.

37. The multimodal tissue scanning system of claim 31, wherein the one or more imaging modalities comprises magnetic resonance imaging (MRI), non-digital or digital x-ray, mammography, computer tomography (CT), and 2D or 3D ultrasound.
